SEQUENCE LISTING (22 sequences)

SEQ ID NO: 1 - DNA, 25 bp, Artificial Sequence
  Synthetic Oligonucleotide
aagcagtggtatcaacgcagagtac

SEQ ID NO: 2 - DNA, 21 bp, Artificial Sequence
  Synthetic Oligonucleotide
tcaccatagttgcgtctcatg

SEQ ID NO: 3 - DNA, 21 bp, Artificial Sequence
  Synthetic Oligonucleotide
gtcaccatagttgcgtctcat

SEQ ID NO: 4 - DNA, 21 bp, Artificial Sequence
  Synthetic Oligonucleotide
cgtcaccatagttgcgtctca

SEQ ID NO: 5 - DNA, 21 bp, Artificial Sequence
  Synthetic Oligonucleotide
tcgtcaccatagttgcgtctc

SEQ ID NO: 6 - DNA, 21 bp, Artificial Sequence
  Synthetic Oligonucleotide
ttcgtcaccatagttgcgtct

SEQ ID NO: 7 - DNA, 8 bp, Artificial Sequence
  Synthetic Oligonucleotide
  misc_difference (1)..(1): 5′ 6-FAM
  misc_feature (4)..(6): n is a, c, g, or t
uuunnnaa

SEQ ID NO: 8 - DNA, 8 bp, Artificial Sequence
  Synthetic Oligonucleotide
  misc_difference (1)..(1): 5′ 6-FAM
  misc_feature (4)..(6): n is a, c, g, or t
uuunnncc

SEQ ID NO: 9 - DNA, 8 bp, Artificial Sequence
  Synthetic Oligonucleotide
  misc_difference (1)..(1): 5′ 6-FAM
  misc_feature (4)..(6): n is a, c, g, or t
uuunnngg

SEQ ID NO: 10 - DNA, 8 bp, Artificial Sequence
  Synthetic Oligonucleotide
  misc_difference (1)..(1): 5′ 6-FAM
  misc_feature (4)..(6): n is a, c, g, or t
uuunnntt

SEQ ID NO: 11 - DNA, 8 bp, Artificial Sequence
  Synthetic Oligonucleotide
  misc_difference (1)..(1): 5′ Cy3
  misc_feature (4)..(6): n is a, c, g, or t
uuunnnac

SEQ ID NO: 12 - DNA, 8 bp, Artificial Sequence
  Synthetic Oligonucleotide
  misc_difference (1)..(1): 5′ Cy3
  misc_feature (4)..(6): n is a, c, g, or t
uuunnnca

SEQ ID NO: 13 - DNA, 8 bp, Artificial Sequence
  Synthetic Oligonucleotide
  misc_difference (1)..(1): 5′ Cy3
  misc_feature (4)..(6): n is a, c, g, or t
uuunnngt

SEQ ID NO: 14 - DNA, 8 bp, Artificial Sequence
  Synthetic Oligonucleotide
  misc_difference (1)..(1): 5′ Cy3
  misc_feature (4)..(6): n is a, c, g, or t
  misc_feature (6)..(6): n is a, c, g, t or u
uuunnntg

SEQ ID NO: 15 - DNA, 8 bp, Artificial Sequence
  Synthetic Oligonucleotide
  misc_difference (1)..(1): 5′ TXR
  misc_feature (4)..(6): n is a, c, g, or t
uuunnnag

SEQ ID NO: 16 - DNA, 8 bp, Artificial Sequence
  Synthetic Oligonucleotide
  misc_difference (1)..(1): 5′ TXR
  misc_feature (4)..(6): n is a, c, g, or t
uuunnnga

SEQ ID NO: 17 - DNA, 8 bp, Artificial Sequence
  Synthetic Oligonucleotide
  misc_difference (1)..(1): 5′ TXR
  misc_feature (4)..(6): n is a, c, g, or t
uuunnnct

SEQ ID NO: 18 - DNA, 8 bp, Artificial Sequence
  Synthetic Oligonucleotide
  misc_difference (1)..(1): 5′ TXR
  misc_feature (4)..(6): n is a, c, g, or t
uuunnntc

SEQ ID NO: 19 - DNA, 8 bp, Artificial Sequence
  Synthetic Oligonucleotide
  misc_difference (1)..(1): 5′ Cy5
  misc_feature (4)..(6): n is a, c, g, or t
uuunnnat

SEQ ID NO: 20 - DNA, 8 bp, Artificial Sequence
  Synthetic Oligonucleotide
  misc_difference (1)..(1): 5′ Cy5
  misc_feature (4)..(6): n is a, c, g, or t
uuunnnta

SEQ ID NO: 21 - DNA, 8 bp, Artificial Sequence
  Synthetic Oligonucleotide
  misc_difference (1)..(1): 5′ Cy5
  misc_feature (4)..(6): n is a, c, g, or t
uuunnngc

SEQ ID NO: 22 - DNA, 8 bp, Artificial Sequence
  Synthetic Oligonucleotide
  misc_difference (1)..(1): 5′ Cy5
  misc_feature (4)..(6): n is a, c, g, or t
uuunnncg

(19) United States
(12) Patent Application Publication
(10) Pub. No.: US 2022/0373544 A1
(43) Pub. Date: Nov. 24, 2022

(54) METHODS AND SYSTEMS FOR DETERMINING CELL-CELL INTERACTION

(71) Applicant: Singleron Biotechnologies Inc., Woodbridge, CT (US)

(72) Inventors: Jing Zhou, Woodbridge, CT (US); Janaki Acharya, Woodbridge, CT (US); Peter Cyrus Torab, Woodbridge, CT (US)

(21) Appl. No.: 17/751,023

(22) Filed: May 23, 2022

Related U.S. Application Data
(60) Provisional application No. 63/192,432, filed on May 24, 2021.

(51) Int. Cl. G01N 33/554; C12N 15/10; C12Q 1/6806

(52) U.S. Cl. CPC G01N 33/554; C12N 15/1065; C12Q 1/6806

(57) ABSTRACT

Disclosed herein include methods, systems, compositions and kits for determining cell-cell interaction, for example, using nucleic acid sequencing. The method can match a cell barcode sequence associated with a cell of a plurality of cells with a cell barcode sequence associated with a partition of a plurality of partitions to identify the partition the cell has originated from in the plurality of partitions. In some embodiments, a pair of interacting cells within a partition are attached with a common cell barcode. The nucleic acid sequences of the pair of interacting cells having the common cell barcode can be tracked to the partition the pair of interacting cells has originated from, where phenotypic observables of the interacting cells can be obtained, for example, using optical imaging, thereby linking cell nucleic acid sequences such as expression profiles to cell functionality and the nature of the cell-cell interaction.

### RELATED APPLICATIONS

The present application claims priority under 35 U.S.C. § 119(e) to U.S. Provisional Application No. 63/192,432, filed May 24, 2021. The content of the related application is incorporated herein by reference in its entirety.

### REFERENCE TO SEQUENCE LISTING

The present application is being filed along with a Sequence Listing in electronic format. The Sequence Listing is provided as a file entitled Sequence_Listing_76PP-328945-US, created May 23, 2022 which is 7 kilobytes in size. The information in the electronic format of the Sequence Listing is incorporated herein by reference in its entirety.

### BACKGROUND

### Field

The present disclosure relates generally to the field of determining cell-cell interaction, for example, determining cell-cell interaction using nucleic acid sequencing.

### Description of the Related Art

Technologies have been developed to investigate single cell genetic information and functionality individually. There is a need for high throughput single cell sequencing technologies that can link the genetic information of single cells to their phenotypic information such as a specific functional feature.

### SUMMARY

Disclosed herein include methods, systems, and devices for determining a profile (e.g., an expression profile, an -omics profile, or a multi-omics profile) of a cell involved in a cell-to-cell interaction. In some aspects, a method of determining an expression profile of a cell involved in a cell-to-cell interaction of interest. In some embodiments, the method comprises partitioning a plurality of first cells and a plurality of second cells into a plurality of partitions. The method can comprise determining an interaction of interest between one first cell of the plurality of first cells and one second cell of the plurality of second cells in a partition of the plurality of partitions. The method can comprise introducing a bead comprising a plurality of barcode molecules into the partition, wherein barcode molecules of the plurality of barcode molecules comprise an identical cell barcode and different unique molecule identifiers (UMIs). The method can comprise barcoding a plurality of sample nucleic acids associated with the first cell and/or the second cell in the partition using the plurality of barcode molecules to generate a plurality of barcoded nucleic acids. The method can comprise pooling a first subset of barcoded nucleic acids of the plurality of barcoded nucleic acids, whereby a second subset of barcoded nucleic acids of the plurality of barcoded nucleic acids are retained in the partition. The method can comprise determining sequences of the pooled barcoded nucleic acids. The method can comprise determining an expression profile from the sequences of the pooled barcoded nucleic acids. The method can comprise determining a sequence of the identical cell barcode of barcoded nucleic acids retained in the partition. The method can comprise matching the sequence of the identical cell barcode of the retained barcoded nucleic acids with a sequence of the identical cell barcode in the sequences of the pooled barcoded nucleic acids. The method can comprise determining an expression profile of the first cell and/or the second cell as the expression profile determined from the sequences of the pooled barcoded nucleic acids. The method can also comprise matching the expression profile of the first cell and/or the second cell determined from the sequences of the pooled barcoded nucleic acids with the interaction of interest.

Disclosed herein include methods of nucleic acid sequencing. In some embodiments, a method of nucleic acid sequencing comprises: (a) partitioning a plurality of first cells and a plurality of second cells into a plurality of partitions. A partition of the plurality of partitions can comprise one first cell of the plurality of first cells and one second cell of the plurality of second cells. The method can comprise: (b) introducing a plurality of barcode molecules to the partition. Each of the plurality of barcode molecules can comprise a first barcode sequence and a second barcode sequence. The first barcode sequences of two barcode molecules of the plurality of barcode molecules can be identical. The method can comprise: (c) barcoding a plurality of sample nucleic acids associated with the first cell and/or the second cell in the partition using the plurality of barcode molecules to generate a plurality of barcoded nucleic acids. The method can comprise: (d) sequencing the plurality of barcoded nucleic acids to obtain nucleic acid sequences of the plurality of barcoded nucleic acids. The method can comprise: (e) determining a location of the partition in which the plurality of sample nucleic acids associated with the first cell and/or the second cell being barcoded is (or was) present using the first barcode sequences determined in the nucleic acid sequences of two barcoded nucleic acids of the plurality of barcoded nucleic acids.

In some embodiments, a method of nucleic acid sequencing comprises: (a) partitioning a plurality of first cells and a plurality of second cells into a plurality of partitions. Each partition of the plurality of partitions can comprise one first cell of the plurality of first cells and one second cell of the plurality of second cells. The method can comprise: for each of one or more of the partitions, (b) introducing a plurality of barcode molecules to the partition. Each of the plurality of barcode molecules can comprise a first barcode sequence and a second barcode sequence. The first barcode sequences of two barcode molecules of the plurality of barcode molecules can be identical. The method can comprise: for each of one or more of the partitions, (c) barcoding a plurality of sample nucleic acids associated with the first cell and/or the second cell in the partition using the plurality of barcode molecules to generate a plurality of barcoded nucleic acids. The method can comprise: for each of one or more of the partitions, (d) sequencing the plurality of barcoded nucleic acids to obtain nucleic acid sequences of the plurality of barcoded nucleic acids. The method can comprise: for each of one or more of the partitions, or for a partition comprising the first cell and the second cell with an interaction of interest, (e) determining a location of the partition in which the plurality of sample nucleic acids associated with the first cell and/or the second cell being barcoded is (or was) present using the first barcode sequences determined in the nucleic acid sequences of two barcoded nucleic acids of the plurality of barcoded nucleic acids.

In some embodiments, introducing the plurality of barcode molecules to the partition occurs before, after, or concurrently with partitioning the plurality of first cells and a plurality of second cells. In some embodiments, the method comprises lysing cells after introducing the plurality of barcode molecules to the partition and before barcoding the plurality of sample nucleic acids. Lysing cells can comprise but not limited to the use of a lysis agent, or the use of a thermal stress, or the use of an electric field, or the use of a mechanical force or stress.

In some embodiments, barcoding the plurality of sample nucleic acids comprises: (c1) extending the plurality of barcode molecules using the plurality of sample nucleic acids as templates to generate the plurality of barcoded nucleic acids comprising single-stranded barcoded nucleic acids hybridized to the plurality of sample nucleic acids in the partition. The method can comprise removing the plurality of sample nucleic acids hybridized to the single-stranded barcoded nucleic acids, such as digesting or hydrolyzing the plurality of sample nucleic acids. Barcoding the plurality of sample nucleic acids can comprise: (c2) generating the plurality of barcoded nucleic acids comprising double-stranded barcoded nucleic acids in the partition using the single-stranded barcoded nucleic acids as templates. Introducing the plurality of barcode molecules to the partition can comprise introducing a plurality of template switching oligonucleotides into the partition. Generating the plurality of barcoded nucleic acids comprising double-stranded barcoded nucleic acids can comprise generating the plurality of barcoded nucleic acids comprising the double-stranded barcoded nucleic acids using the plurality of template switching oligonucleotides in the partition.

In some embodiments, introducing the plurality of barcode molecules to the partition comprises: introducing a plurality of extension primers to the partition. Barcoding the plurality of sample nucleic acids can comprise: (c1) extending the plurality of extension primers using the plurality of sample nucleic acids as templates and the plurality of barcode molecules as template switching oligonucleotides to generate a plurality of single-stranded barcoded nucleic acids of the plurality of barcoded nucleic acids that are hybridized to the plurality of sample nucleic acids. Barcoding the plurality of sample nucleic acids can comprise: (c2) generating a plurality of double-stranded barcoded nucleic acids of the plurality of barcoded nucleic acids using the plurality of single-stranded barcoded nucleic acids as templates. The method comprises removing the plurality of sample nucleic acids hybridized to the plurality of single-stranded barcoded nucleic acids, such as digesting or hydrolyzing the plurality of sample nucleic acids. In some embodiments, the plurality of sample nucleic acids comprises poly-adenylated messenger ribonucleic acid (mRNA). The extension primers can comprise a poly(dT) sequence.

In some embodiments, the method comprises denaturing the double-stranded barcoded nucleic acids of the plurality of barcoded nucleic acids to generate single-stranded barcoded nucleic acids. The single-stranded barcoded nucleic acids can comprise a single-stranded barcoded nucleic acid retained in the partition and a single-stranded barcoded nucleic acid released from the retained single-stranded barcoded nucleic acid when the double-stranded nucleic acids are denatured.

In some embodiments, the plurality of sample nucleic acids comprises deoxyribonucleic acid (DNA). The plurality of sample nucleic acids can comprise genomic DNA (gDNA). The plurality of sample nucleic acids can comprise ribonucleic acid (RNA).

In some embodiments, wherein barcoding the plurality of sample nucleic acids comprises a reverse transcription reaction, and wherein the plurality of barcoded nucleic acids comprises complementary deoxyribonucleic acid (cDNA). In some embodiments, a barcode molecule of the plurality of barcode molecules comprises a target binding sequence capable of hybridizing to the plurality of sample nucleic acids. In some embodiments, a barcode molecule of the plurality of barcode molecules comprises a target binding sequence capable of hybridizing to a specific sample nucleic acid of the plurality of sample nucleic acids associated with the first cell and/or second cell. The target binding sequence can comprise a poly(dT) sequence or a specifically designed target binding sequence. Barcoding the plurality of sample nucleic acids can comprise hybridizing the poly(dT) sequence of the target binding sequence to a poly(A) tail of a sample nucleic acid of the plurality of sample nucleic acids associated with the first cell and/or the second cell. Barcoding the plurality of sample nucleic acids can comprise hybridizing the target binding region comprising the specifically designed target binding sequence to a sample nucleic acid of the plurality of sample nucleic acids associated with the first cell and/or the second cell. The sample nucleic acid can comprise a complementary sequence of the specifically designed target binding sequence such that the target binding region comprising the specifically designed target binding sequence is capable of hybridizing to the sample nucleic acid.

In some embodiments, the method comprises pooling barcoded nucleic acids of the plurality of barcoded nucleic acids, after barcoding the plurality of sample nucleic acids and before sequencing the plurality of barcoded nucleic acids, to obtain pooled barcoded nucleic acids. The pooled barcoded nucleic acids can comprise single-stranded barcoded nucleic acids released from nucleic acids retained in the partition. The pooled barcoded nucleic acids can comprise double-stranded barcoded nucleic acids. The method can comprise generating the double-stranded barcoded nucleic acids from the single-stranded barcoded nucleic acids (e.g., the barcoded nucleic acids retained in the partition and/or released from the barcoded nucleic acids retained in the partition).

In some embodiments, sequencing the plurality of barcoded nucleic acids comprises sequencing the pooled barcoded nucleic acids to obtain nucleic acid sequences of the pooled barcoded nucleic acids. The nucleic acid sequences of each pooled barcoded nucleic acid comprise a first sequence (or a reverse complement thereof) and a second barcode sequence (or a reverse complement thereof) and a sequence of a sample nucleic acid (e.g., the full sequence or a subsequence of the sample nucleic acid) associated with the first cell and/or the second cell (or a reverse complement thereof).

In some embodiments, the method comprises fragmenting the pooled barcoded nucleic acids, before sequencing the plurality of barcoded nucleic acids, to generate fragmented barcoded nucleic acids (e.g., for sequencing library generation). Fragmenting the pooled barcoded nucleic acids can comprise enzymatic fragmentation (e.g., non-specific or specific restriction enzyme digestion using one or more restriction enzymes), physical fragmentation (e.g., sonication, nebulization, shearing), or a combination thereof.

In some embodiments, the method comprises performing a polymerase chain reaction in bulk, subsequent to pooling barcoded nucleic acids of the plurality of barcoded nucleic acids, on the pooled barcoded nucleic acids (and/or fragmented pooled barcoded nucleic acids), thereby generating amplified barcoded nucleic acids. Performing the polymerase chain reaction in bulk can be subsequent to fragmenting the pooled barcoded nucleic acids. The amplified barcoded nucleic acids can comprise a sequence for attaching the amplified barcoded nucleic acids to a flow well (e.g., for sequencing-by-synthesis). The sequence for attaching the amplified barcoded nucleic acids to the flow well can comprise a P5 sequence (or a portion thereof) and/or a P7 sequence (or a portion thereof). The amplified barcoded nucleic acids can comprise a sequencing primer sequence (or a sequencing primer binding sequence). The sequencing primer sequence can comprise a Read 1 sequence or a Read 2 sequence or a portion thereof. Sequencing the plurality of barcoded nucleic acids can comprise sequencing the amplified barcoded nucleic acids, or products thereof.

In some embodiments, the plurality of barcoded nucleic acids comprises barcoded nucleic acids retained in the partition. Sequencing the plurality of barcoded nucleic acids can comprise sequencing the barcoded nucleic acids retained in the partition. Sequencing the barcoded nucleic acids retained in the partition can comprise determining the first barcode sequences of the barcoded nucleic acids retained in the partition. The barcoded nucleic acids retained in the partition can comprise single-stranded barcoded nucleic acids. Sequencing the barcoded nucleic acids retained in the partition can comprise: determining the first barcode sequences of the barcoded nucleic acids retained in the partition using oligonucleotide probes comprising fluorescent labels. In some embodiments, sequencing the barcoded nucleic acids retained in the partition comprises: determining the first barcode sequences of the barcoded nucleic acids retained in the partition using sequencing-by-ligation. Determining the first barcode sequences of the barcoded nucleic acids retained in the partition using sequencing-by-ligation can comprise one or more cycles of introducing a sequencing primer capable of hybridizing to the barcoded nucleic acids retained in the partition. Determining the first barcode sequences of the barcoded nucleic acids retained in the partition using sequencing-by-ligation can comprise one or more cycles of extending the sequencing primer using the barcoded nucleic acids retained in the partition as templates to generate a plurality of extended sequencing primers comprising the first barcode sequences, or a portion thereof, of the barcoded nucleic acids retained in the partition. A different sequencing primer can be used in different cycles. In some embodiments, the method comprises introducing a plurality of oligonucleotide probes each comprising a fluorescent label. The plurality of oligonucleotide probes can be, for example, octamer probes.

In some embodiments, the two barcoded nucleic acids of the plurality of barcoded nucleic acids comprise a retained barcoded nucleic acid of the retained barcoded nucleic acids and a pooled barcoded nucleic acid of the pooled barcoded nucleic acids. The first barcode sequence of the retained barcoded nucleic acid and the first barcode sequence of the pooled barcoded nucleic acid can be identical.

In some embodiments, determining the location of the partition in which the plurality of sample nucleic acids associated with the first cell and/or the second cell being barcoded is (or was) present comprises: matching the first barcode sequence of the barcoded nucleic acids retained in the partition determined and the first barcode sequence of the pooled barcoded nucleic acids in the nucleic acid sequences obtained. The method can comprise: recording a location of the partition comprising the first cell and the second cell, or characteristics of the first cell and/or the second cell therein, to provide a recorded location of the partition, or characteristics of the first cell and/or the second cell therein. The method can comprise: linking the recorded location of the partition comprising the first cell and the second cell, or the characteristics of the first cell and/or the second cell therein, with the first barcode sequence of the barcoded nucleic acids retained in the partition determined. The recorded location of the partition and the first barcode sequence of the pooled barcoded nucleic acids associated with the first cell and/or the second cell can thereby be linked. Recording the location of the partition comprising the first cell and the second cell, or characteristics of the first cell and the second cell therein, can comprise optically imaging the partition, or the first cell and the second cell therein.

In some embodiments, the characteristics of the first cell and/or the second cell therein comprises a phenotypic feature of the first cell and/or the second cell. The phenotypic feature can comprise cell viability, cell activity, a cell size, a nucleus size, cell morphology, nucleus morphology, a surface marker expression level, a protein expression level (e.g., a fluorescence protein expression level), a signaling dynamics, a signaling behavior, or a combination thereof. In some embodiments, the first cell interacts with the second cell. An interaction between the first cell and the second cell can be of interest. In some embodiments, the interaction between the first cell and the second cell that is of interest comprises an increase in cell proliferation, an increase in cell viability, an increase in cell movement, an increase in cell differentiation, an expression of a specific protein or an elevation of its expression level, a secretion of a specific cytokine or elevation of its secretion amount, or a combination thereof. In some embodiments, the interaction between the first cell and the second cell that is of interest comprises an inhibition in cell movement, a reduction of cell proliferation, a decrease in cell viability, an inhibition in cell differentiation, a decrease of protein expression, a decrease of cytokine secretion, or a combination thereof In some embodiments, the first cell is a T cell. The second cell can be a B cell (e.g., a B cell that is presenting an antigen, such as a specific antigen). In some embodiments, a partition comprises at most one T cell and one or more B cells. The interaction between the first cell and the second cell can result in functionalization of the first cell. The interaction between the first cell and the second cell can result in death of the second cell. Functionalizing of the first cell can result in death of the second cell. In some embodiments, the first cell and/or the second cell is an immune cell, optionally wherein the immune cell is a neutrophil, an eosinophil, a basophil, a mast cell, a monocyte, a macrophage, a dendritic cell, a natural killer cell, a lymphocyte, a B cell, a T cell, or a combination thereof.

In some embodiments, the interaction between the first cell and the second cell that is of interest comprises (or results in) a change in a profile of the first cell and/or the second cell. The profile can comprise a transcriptomics profile, optionally wherein the profile comprises a mutli-omics profile, optionally wherein the multi-omics profile comprises a genomics profile, a proteomics profile, a transcriptomics profile, an epigenomics profile, a metabolomics profile, a chromatics profile, a protein expression profile, a cytokine secretion profile, or a combination thereof. In some embodiments, the method comprises determining the profile of the first cell and/or the second cell using the nucleic acid sequences (for example, using the second barcode sequences and sequences of the sample nucleic acids (or portions thereof) present in the nucleic acid sequences) of the plurality of barcoded nucleic acids. The expression profile of the first cell and/or the second cell in the partition determined can be different from an expression profile of the first cell or the second cell alone. In some embodiments, the method comprises: linking (1) the profile of the first cell and/or the second cell in the partition determined with (2a) the characteristics of the first cell and/or the second cell in the partition, (2b) the phenotypic feature of the first cell and/or the second cell, and/or (2c) the interaction of interest between the first cell and the second cell using (i) the first barcode sequence of the retained barcoded nucleic acid determined and (ii) the first barcode sequence of the pooled barcoded nucleic acid in the sequencing data. In some embodiments, the method comprises: culturing the first cell and the second cell in the partition subsequent to partitioning the plurality of first cells and the plurality of second cells and prior to barcoding the plurality of sample nucleic acids. The interaction of interest can occur when culturing the first cell and the second cell.

In some embodiments, the plurality of barcode molecules is associated with a bead. The plurality of barcode molecules can be attached to (e.g., reversibly attached to, covalently attached to, or irreversibly attached to) a bead. In some embodiments, the bead is a solid bead. In some embodiments, the bead is a gel bead. The gel bead is degradable upon application of a stimulus. The stimulus comprises a thermal stimulus, a chemical stimulus, a biological stimulus, a photo-stimulus, or a combination thereof. In some embodiments, the bead is a magnetic bead (e.g., comprising paramagnetic material). The bead can be retained in the partition by an external magnetic field during one, one or more, or each of the steps of the method. In some embodiments, the bead has a dimension about 10 μm to about 100 μm. In some embodiments, the partition of the plurality of partitions comprises at most one bead. At least 80% of the plurality of partitions each comprises at most one bead.

In some embodiments, the first barcode sequences are cell barcodes for identifying the plurality of barcoded nucleic acids which originate from the first cell and/or the second cell. The cell barcodes each can be 2-40 nucleotides in length. In some embodiments, the second barcode sequences are unique molecule identifiers (UMIs) for identifying molecular origins of the plurality of barcoded nucleic acids. The UMIs can be 2-40 nucleotides in length. The second barcode sequences of two barcode molecules of the plurality of barcode molecules can be different. The second barcode sequences of two barcode molecules of the plurality of barcode molecules can be identical.

In some embodiments, each of the plurality of barcode molecules comprises a primer sequence. The primer sequence can be a sequencing primer sequence (or a sequencing primer binding sequence). The sequencing primer sequence can be a Read 1 sequence, a Read 2 sequence, or a portion thereof. In some embodiments, a barcode molecule of the plurality of barcode molecules comprises a template switching oligonucleotide. In some embodiments, each of the plurality of barcode molecules comprises a PCR sequence.

In some embodiments, the plurality of partitions comprises about 30,000 to 50,000 partitions. In some embodiments, the plurality of partitions comprises a plurality of microwells. The plurality of microwells can be in a microfluidic device. The microfluidic device can comprise an inlet port in fluid communication with the plurality of microwells. The microfluidic device can comprise an outlet port in fluid communication with the plurality of microwells. In some embodiments, partitioning the plurality of first cells and a plurality of second cells comprises flowing a solution comprising the plurality of first cells and/or the plurality of second cells into the plurality of microwells via the inlet port. Flowing the solution comprising the plurality of first cells and/or the plurality of second cells comprises flowing a solution comprising the plurality of first cells and a solution comprising the plurality of second cells concurrently or sequentially. In some embodiments, introducing the plurality of barcode molecules comprises flowing a solution comprising the plurality of barcode molecules into the plurality of microwells via the inlet port.

In some embodiments, barcoding the plurality of sample nucleic acids comprises flowing reverse transcription reagents into the plurality of microwells via the inlet port, thereby generating partially single-stranded/partially double-stranded barcoded nucleic acids of the plurality of barcoded nucleic acids hybridized to sample nucleic acids of the plurality of sample nucleic acids. The partially single-stranded/partially double-stranded barcoded nucleic acids hybridized to sample nucleic acids can be separated by denaturation (e.g., heat denaturation or chemical denaturation using for example, sodium hydroxide) to generate single-stranded barcoded nucleic acids of the plurality of barcoded nucleic acids in the partition. Barcoding the plurality of sample nucleic acids comprises flowing polymerase chain reaction reagents (or extension reagents) into the plurality of microwells via the inlet port subsequent to the reverse transcription, thereby generating the plurality of barcoded nucleic acids comprising double-strand barcoded nucleic acids in the partition.

In some embodiments, the partition of the plurality of partitions comprises one first cell, two first cells, or two or more first cells. The partition of the plurality of partitions comprises one second cell, two second cells, or two or more second cells. At most 25% of the plurality of partitions is each occupied with one first cell of the plurality of first cells and/or one second cell of the plurality of second cell.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1A is a schematic diagram showing a non-limiting example of mRNA barcoding and sequencing library preparation.

FIG. 1B is a schematic diagram showing another non-limiting example of mRNA barcoding and sequencing library preparation.

FIG. 2 is a schematic diagram showing a non-limiting workflow for determining cell-cell interaction.

FIG. 3 shows a process schematic of an embodiment disclosed herein.

FIG. 4A demonstrates an exemplary microwell array loaded with about 10% single cell coverage of the microwells.

FIG. 4B demonstrates an exemplary microwell array loaded with microbeads to ensure only one microbead in one microwell.

FIG. 5A shows a non-limiting exemplary trace showing the quality of the purified cDNA solution after PCR amplification.

FIG. 5B shows in a non-limiting exemplary trace showing the quality of the purified libraries.

FIG. 6A shows in one exemplary plot the UMI count vs. cell barcode number.

FIG. 6B shows in three exemplary violin plots of the gene number (left plot), UMI number (middle plot), and mitochondria percentage distribution (right plot) of determined cells.

FIG. 7 illustrates a three round of sequencing for Primer N-0 in sequencing-by-ligation.

FIG. 8 illustrates a decoding process of the 12 bp cell barcode sequence for five sequencing primers in sequencing-by-ligation.

FIG. 9 shows, in a non-limiting example, the microbeads with barcodes in a microwell array before adding the sequencing primer N-0 (in panels A and B) and after adding the sequencing primer N-0 (in panels C and D).

FIG. 10 shows, in a non-limiting example, the microbeads with barcodes in a microwell array after adding a mixture of four sequencing oligos with fluorescent dye of GFP, RFP, Texas Red, and Cy5 following the addition of sequencing primer N-0 during an on-chip sequencing process.

FIG. 11 shows, in a non-limiting example, the microbeads with barcodes in a microwell array after cleavage of the sequencing oligos with fluorescent dye of GFP, RFP, Texas Read and Cy5.

FIG. 12 shows an exemplary schematic of an automatic flow control system for on-chip sequencing.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Biological events often involve cell-cell interactions (e.g. immune response). Immunotherapy depends on the activation or suppression of the immune system (e.g. immune cells to attack or to be silent to target cells) to treat patients. Functionality of cells is an important characteristic for cell therapeutic drug discovery. However, due to the cell heterogeneity, even cells from the same source, going through the same desirable engineering or bio-modification process and presenting expected biological markers may have distinct functionality and result in different therapeutic outcomes in patients. Therefore, for cell therapeutic drug discovery, it is important to screen each cell individually with specific functionality as well as biological marker development and genetic information discovery.

Technologies have been developed to investigate single cell biologic marker development, genetic information, and functionality individually. For example, flow cytometry can distinguish and sort single cells with different biomarkers based on different fluorescent signals. Single cell sequencing technology has been used to interrogate the genetic information of single cells. Microwell arrays, microfluidic traps, and microdroplets are used to evaluate the cytotoxicity of single cells by pairing attack cells with target cells. However, because cells are randomly distributed into microwells, existing technologies do not correlate the measured biomarkers and genetic information with cells having specific functionality, especially in a high throughput manner.

Disclosed herein include embodiments of a method of determining an expression profile of a cell involved in a cell-cell interaction of interest using, for example, nucleic acid sequencing. The method can match the cell barcode sequence associated with a cell of a plurality of cells with the cell barcode sequence associated with a partition in a plurality of partitions to identify the location of the partition the cell has originated from in the plurality of partitions. In some embodiments, by attaching common cell barcodes to a pair of interacting cells within a given partition, the resulting sequences of the pair of interacting cells having the common cell barcodes can be tracked to that partition where phenotypic observables (e.g. cellular metabolism, cell cycle states, cell signaling, cell viability etc.) of the interacting cells can be obtained, thereby linking cell nucleic acid sequences such as expression profiles to cell functionality and the nature of the cell-cell interaction. The method can be used to investigate cell functionality of various cell types including but not limited to immune cells (e.g. T cells, B cells, or natural killer cells), fibroblast cells, stem cells, cancer cells, or any cells the functionality of which can be affected by the presence of other cells.

Disclosed herein include embodiments of a method of determining an expression profile of a cell involved in a cell-to-cell interaction of interest. The method comprises partitioning a plurality of first cells and a plurality of second cells into a plurality of partitions. The method can comprise determining an interaction of interest between one first cell of the plurality of first cells and one second cell of the plurality of second cells in a partition of the plurality of partitions. The method can comprise introducing a bead comprising a plurality of barcode molecules into the partition, wherein barcode molecules of the plurality of barcode molecules comprise an identical cell barcode and different unique molecule identifiers (UMIs). The method can comprise barcoding a plurality of sample nucleic acids associated with the first cell and/or the second cell in the partition using the plurality of barcode molecules to generate a plurality of barcoded nucleic acids. The method can comprise pooling a first subset of barcoded nucleic acids of the plurality of barcoded nucleic acids, whereby a second subset of barcoded nucleic acids of the plurality of barcoded nucleic acids are retained in the partition. The method can comprise determining sequences of the pooled barcoded nucleic acids. The method can comprise determining an expression profile from the sequences of the pooled barcoded nucleic acids. The method can comprise determining a sequence of the identical cell barcode of the barcoded nucleic acids retained in the partition. The method can comprise matching the sequence of the identical cell barcode of the barcoded nucleic acids retained in the partition and a sequence of the identical cell barcode in the sequences of the pooled barcoded nucleic acids. The method can comprise determining an expression profile of the first cell and/or the second cell as the expression profile determined from the sequences of the pooled barcoded nucleic acids. The method can further comprise matching the expression profile of the first cell and/or the second cell determined from the sequences of the pooled barcoded nucleic acids with the interaction of interest.

Disclosed herein include embodiments of a method of nucleic acid sequencing. In some embodiments, the method comprises partitioning a plurality of first cells and a plurality of second cells into a plurality of partitions, wherein a partition of the plurality of partitions comprises one first cell of the plurality of first cells and one second cell of the plurality of second cells. The method can comprise introducing a plurality of barcode molecules to the partition, wherein each of the plurality of barcode molecules comprises a first barcode sequence and a second barcode sequence, and wherein the first barcode sequences of two barcode molecules of the plurality of barcode molecules are identical. Introducing a plurality of barcode molecules to the partition can occur before, after, or concurrently with partitioning a plurality of first cells and a plurality of second cells. The method can comprise barcoding a plurality of sample nucleic acids associated with the first cell and/or the second cell in the partition using the plurality of barcode molecules to generate a plurality of barcoded nucleic acids. The method can comprise sequencing the plurality of barcoded nucleic acids to obtain nucleic acid sequences of the plurality of barcoded nucleic acids. The method can also comprise determining a location of the partition in which the plurality of sample nucleic acids associated with the first cell and/or the second cell being barcoded is (or was) present using the first barcode sequences of two barcoded nucleic acids of the plurality of barcoded nucleic acids detected in the nucleic acid sequences. The method can also comprise lysing cells after introducing a plurality of barcode molecules to the partition and before barcoding a plurality of sample nucleic acids to release the contents of the cells (e.g. sample nucleic acids). The method can also comprise determining an expression profile of the first cell and/or the second cell wherein the first cell and the second cell interact with each other in a partition and matching the determined expression profile of the first cell and/or the second cell with an interaction of interest between the first cell and the second cell.

Disclosed herein include embodiments of a method of determining nucleic acid sequences. In some embodiments, the method comprises partitioning a plurality of first cells and a plurality of second cells into a plurality of partitions, wherein each of partitions of the plurality of partitions comprises one first cell of the plurality of first cells and one second cell of the plurality of second cells. For each of one or more of the partitions, the method can comprise introducing a plurality of barcode molecules to the partition, wherein each of the plurality of barcode molecules comprises a first barcode sequence and a second barcode sequence, and wherein the first barcode sequences of two barcode molecules of the plurality of barcode molecules are identical. The method can comprise sequencing the plurality of barcoded nucleic acids to obtain nucleic acid sequences of the plurality of barcoded nucleic acids. The method can also comprise determining a location of the partition in which the plurality of sample nucleic acids associated with the first cell and/or the second cell being barcoded is (or was) present using the first barcode sequences determined in the nucleic acid sequences of two barcoded nucleic acids of the plurality of barcoded nucleic acids.

### Determining Cell-Cell Interaction

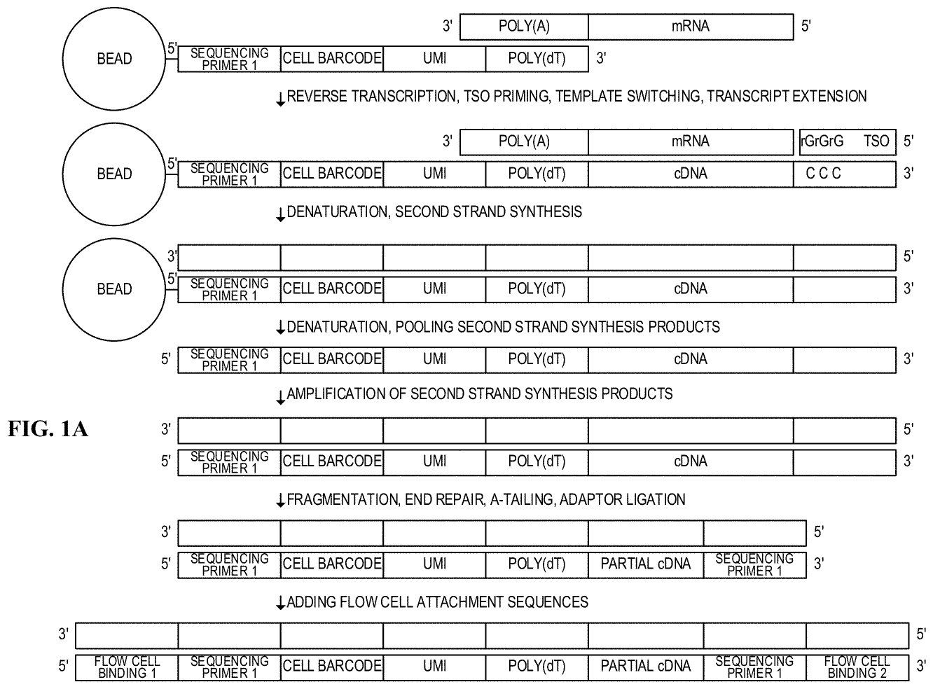

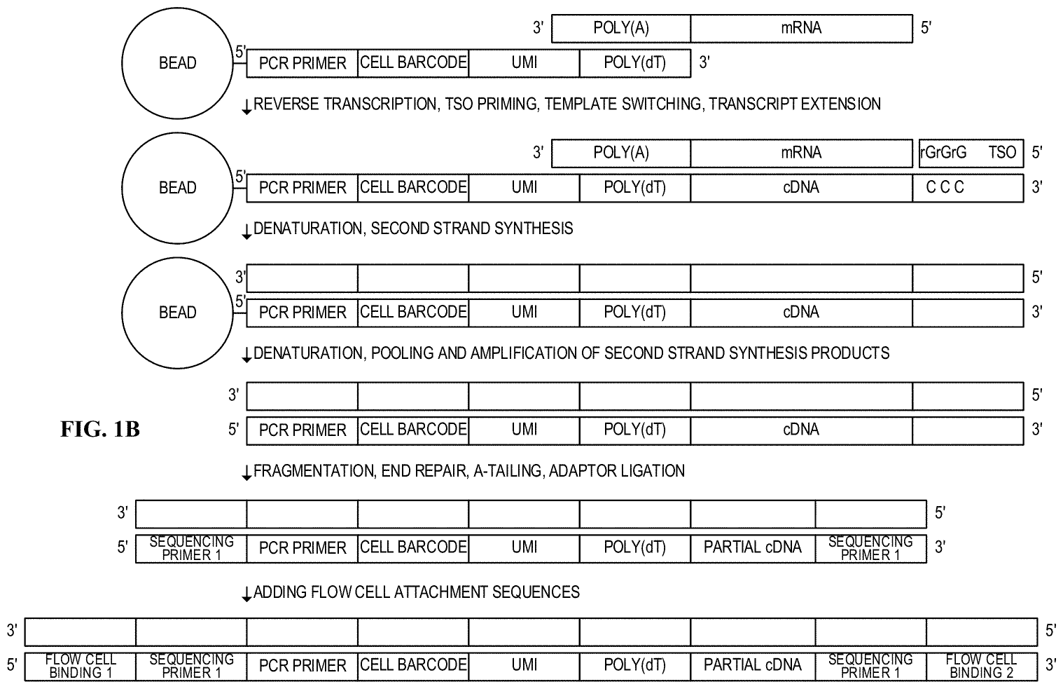

Disclosed herein include methods, compositions, and kits for determining cell-cell interactions, for example, using nucleic acid sequencing, such as RNA sequencing. Two cells can be distributed into a partition (e.g., a microwell). The interaction between the two cells can be determined using, for example, optical imaging. The interaction between the two cells can be of interest. Referring to FIGS. 1A-1B, nucleic acids (e.g., mRNA) of one or both of the cells can be barcoded using barcode molecules (e.g., barcode molecules attached to a bead, such as a magnetic bead) with a partition-specific barcode (or a cell-specific barcode) and a unique molecule identifier (UMI) to generate barcoded nucleic acids (e.g., barcoded cDNAs or products thereof). The partition-specific barcode sequences of barcoded nucleic acids can be identical. Some barcoded nucleic acids (e.g., second strand synthesis products) can be pooled. Some barcoded nucleic acids (e.g., first strand synthesis products attached to a bead) can remain or be retained in the partition. The partition-specific barcode sequences of barcoded nucleic acids retained in the partition can be determined using, for example, fluorescent probes. The pooled barcoded nucleic acids can be sequenced. The sequencing data (e.g., the UMI counts of nucleic acids barcoded) can be processed to determine a nucleic acid profile (e.g., an mRNA expression profile) of one or both of the cells and the partition-specific barcode sequence of the partition in which the cells were present. The sequencing data can include partition-specific barcode sequences. The nucleic acid profile can include or be associated with (e.g., linked to) the partition-specific barcode sequences in the sequencing data. The nucleic acid profile can be linked to the partition using the partition-specific barcode sequences. For example, the partition-specific barcode sequences of the pooled barcoded nucleic acids pooled in the sequencing data can be matched with the partition-specific barcode sequences of barcoded nucleic acids retained in the partition. The nucleic acid profile can in turn be linked to the cell-cell interaction of interest determined.

Introducing Cells and Barcode Molecules into Partitions

Disclosed herein include a method of determining cell-cell interaction using nucleic acid sequencing. Disclosed herein also include a method of nucleic acid sequencing. In some embodiments, the method can comprise introducing a plurality of first cells and/or a plurality of second cells and/or a plurality of barcode molecules into a plurality of partitions. The introduction of a plurality of first cells and/or a plurality of second cells and/or a plurality of barcode molecules (alone or attached to beads) can be performed using partitioning.

As used herein, the term “partitioning” refers to introducing particles (e.g., cells, or beads) into vessels (e.g., microwells, droplets) that can be used to sequester or separate one particle from another. Such vessels are referred to using the noun “partition.” A partition can include two or more particles of the same type or different types.

Partitioning can be performed using a variety of methods known to a person skilled in the art, for example, using microfluidics, wells, microwells, multi-well plates, multi-well arrays, dispensing, dilution, droplets and the like. For example, the cells, barcode molecules, and/or beads can be diluted and dispensed across a plurality of partitions via the use of flow channels in a microwell array.

A partition as used herein can refer to a part, a portion, or a division sequestered from the rest of the parts, portions, or divisions. A partition can be formed through the use of wells, microwells, multi-well plates, microwell arrays, microfluidics, dilution, dispensing, droplets, or any other means of sequestering one fraction of a sample from another. In some embodiments, a partition is a droplet or a microwell.

In some embodiments, the method can comprise partitioning a plurality of first cells and a plurality of second cells into a plurality of partitions, wherein a partition of the plurality of partitions comprises one first cell of the plurality of first cells and one second cell of the plurality of second cells. The first cell and the second cell can be involved in a cell-cell interaction. The plurality of first cells and the plurality of second cells can be partitioned together (e.g. co-partitioned) or separately.

In some embodiments, the method can also comprise partitioning a plurality of barcode molecules into the plurality of partitions. For example, the plurality of barcode molecules can be attached to beads and the method can comprise partitioning a plurality of beads with the plurality of barcode molecules attached thereon into the plurality of partitions. Partitioning the plurality of beads into the plurality of partitions can be performed before, after, or concurrently with partitioning the plurality of first cells and/or the plurality of second cells.

In some embodiments, a plurality of first cells, a plurality of second cells, and/or a plurality of beads with a plurality of barcode molecules attached thereon can be co-partitioned by combining the plurality of first cells, the plurality of second cells, and/or the plurality of beads with a plurality of barcode molecules attached thereon to form a mixture that can be then partitioned into a plurality of partitions.

In some embodiments, partitioning a plurality of first cells, a plurality of second cells, and/or a plurality of beads with a plurality of barcode molecules attached thereon can be performed through the use of fluid flow in microwell array. For example, the partitioning can comprise flowing one or more solutions comprising a plurality of first cells, a plurality of second cells, and/or a plurality of beads with a plurality of barcode molecules attached thereon, sequentially or concurrently in a mixture, into the plurality of microwells via the inlet port.

In some embodiments, introducing the plurality of barcode molecules into the plurality of partitions can be performed without using a bead. In some embodiments, the plurality of barcode molecules can be introduced into the partitions (e.g. microwells) by attaching or synthesizing the plurality of barcode molecules onto the surface of the partitions.

In some embodiments, attaching or synthesizing the plurality of barcode molecules onto the surface of the partitions can involve a ligation step. In some embodiments, synthesizing the plurality of barcode molecules can comprise ligating two smaller oligonucleotides together to generate a plurality of barcode molecules each having a pre-designed sequence. For example, a primer can be attached to the surface of a partition which can hybridize to a primer binding site of an oligonucleotide that also contains a template nucleotide sequence. The primer can then be extended by a primer extension reaction or other amplification reaction, and an oligonucleotide complementary to the template oligonucleotide can thereby be attached to the surface of the partition.

In some embodiments, the surface of the partitions (e.g. microwells) are pre-functionalized with a chemical moiety to facilitate the attachment of barcode molecules. The attachment of the barcode molecules can occur through the interaction between two members of a binding pair, one attached to the surface of the partitions and the other comprised in or conjugated to the barcode molecules, or a portion thereof. For example, the surface of the microwell can be coated with a moiety (e.g. a member of a binding pair) capable of binding with another moiety (e.g. the other member of the binding pair) of the barcode molecule, such that the binding of the two moieties results in the attachment of the barcode molecule or a portion thereof to the microwell. For example, the surface of the microwell can be coated with streptavidin. The biotinylated barcode molecules can be attached to the surface of the microwell via streptavidin-biotin interaction.

In some embodiments, the surface of the partitions (e.g. microwells) can be modified to enhance its chemical reactivity and facilitate the oligonucleotide attachment, such as, by treating the microwells with oxygen plasma, corona discharges, and ultraviolet/ozone (UVO) as will be understood by a person skilled in the art.

Partitions

A partition can be sized to fit at most one bead (and a first cell and/or a second cell), not two beads. A size or dimension (e.g., length, width, depth, radius, or diameter) of a partition can be different in different embodiments. In some embodiments, a size or dimension of one, one or more, or each, of the plurality of partitions is, is about, is at least, is at least about, is at most, or is at most about, 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, a size or dimension of one, one or more, or each, of the plurality of partitions is about 1 nm to about 100 μm.

The volume of one, one or more, or each, of the plurality of partitions can be different in different embodiments. The volume of one, one or more, or each, of the plurality of partitions can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 10000000 nm³, 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm³, or a number or a range between any two of these values. The volume of one, one or more, or each, of the plurality of partitions can be, be about, be at least, be at least about, be at most, or be at most about, 1 nanolieter (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, one or more, or each, of the plurality of partitions is about 1 nm³ to about 1000000 μm³.

The number of partitions can be different in different embodiments. In some embodiments, the number of partitions is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number of partitions can be at least 1000 partitions.

The percentage of the plurality of partitions comprising one or two cells (a first cell and/or a second cell) and a single bead can be different in different embodiments. In some embodiments, the percentage of the plurality of partitions comprising one or two cells (a first cell and/or a second cell) and a single bead is, is about, is at least, is at least about, is at most, or is at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 10% of partitions of the plurality of partitions comprise a first cell of the plurality of first cells and/or a second cell of the plurality of second cells and a single bead of the plurality of beads.

The percentage of the plurality of partitions comprising no cell can be different in different embodiments. In some embodiments, the percentage of the plurality of partitions comprising no cell is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 50% of partitions of the plurality of partitions can comprise no cell of the plurality of cells.

The percentage of the plurality of partitions comprising more than two cells can be different in different embodiments. In some embodiments, the percentage of the plurality of partitions comprising more than two cells is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at most 10% of partitions of the plurality of partitions can comprise more than two cells of the plurality of cells.

Microwells

In some embodiments, the partition is a microwell and the plurality of partitions comprise a plurality of microwells in a microwell array. The term “microwell,” as used herein, generally refers to a well with a volume of less than 1 mL. A microwell array can contain a number of microwells arranged in rows and columns. The size and spacing of the microwells may vary depending on different applications. A location of a microwell in a microwell array can be identified by its unique address describing its row and column position within the microwell array.

The microwell array comprising a plurality of microwells can be formed from any suitable material as will be understood by a person skilled in the art. In some embodiments, a microwell array comprising a plurality of microwells can be formed from a material selected from the group consisting of silicon, glass, ceramic, elastomers such as polydimethylsiloxane (PDMS) and thermoset polyester, thermoplastic polymers such as polystyrene, polycarbonate, poly(methyl methacrylate) (PMMA), poly-ethylene glycol diacrylate (PEGDA), Teflon, polyurethane (PU), composite materials such as cyclic-olefin copolymer, and combinations thereof.

In some embodiments, the microwell array can comprise an inlet port in fluid communication with the plurality of microwells. The microwell array can also comprise an outlet port in fluid communication with the plurality of microwells. Microwells can be introduced with samples, free reagents, and/or reagents encapsulated in microcapsules. The reagents can comprise restriction enzymes, ligase, polymerase, fluorophores, oligonucleotide barcodes, oligonucleotide probes, adapters, buffers, dNTPs, ddNTPs, and other reagents required for performing the methods described herein. Samples and reagents can flow from the inlet port through a flow channel to deliver to the microwell array, and the waste can be pushed out from the outlet port and removed.

Sample Nucleic Acids and Cells

The plurality of cells (e.g. the first cells and second cells) introduced into a plurality of partitions can be obtained from any organism of interest such as Monera (bacteria), Protista, Fungi, Plantae, and Animalia Kingdoms. A cell can be a mammalian cell, and particularly a human cell such as T cells, B cells, natural killer cells, stem cells, cancer cells, or any cells the functionality of which can be affected by the presence of other cells (e.g. cells involved in cell-cell interaction). In some embodiments, the first cell and/or the second cell can be an immune cell. For example, the cell can a neutrophil, an eosinophil, a basophil, a mast cell, a monocyte, a macrophage, a dendritic cell, a natural killer cell, a lymphocyte, a B cell, a T cell, or a combination thereof.

Cells described herein can be obtained from a cell sample. A cell sample comprising cells can be obtained from any source including a clinical sample and a derivative thereof, a biological sample and a derivative thereof, a forensic sample and a derivative thereof, and a combination thereof. A cell sample can be collected from any bodily fluids including, but not limited to, blood, urine, serum, lymph, saliva, anal, and vaginal secretions, perspiration and semen of any organism. A cell sample can be products of experimental manipulation including purification, cell culturation, cell isolation, cell separation, cell quantification, sample dilution, or any other cell sample processing approaches. A cell sample can be obtained by dissociation of any biopsy tissues of any organism including, but not limited to, skin, bone, hair, brain, liver, heart, kidney, spleen, pancreas, stomach, intestine, bladder, lung, esophagus.

The sample nucleic acids associated with a plurality of first cells and/or a plurality of second cells can comprise deoxyribonucleic acid (DNA), ribonucleic acid (RNA), and/or any combination or hybrid thereof. The sample nucleic acids can be single-stranded or double-stranded, or contain portions of both double-stranded or single-stranded sequences. The sample nucleic acids can contain any combination of nucleotides, including uracil, adenine, thymine, cytosine, guanine, inosine, xanthine, hypoxanthine, isocytosine, isoguanine and any nucleotide derivative thereof. As used herein, the term “nucleotide” may include naturally occurring nucleotides and nucleotide analogs, including both synthetic and naturally occurring species. The sample nucleic acids can be genomic DNA (gDNA), mitochondrial DNA (mtDNA), messenger RNA (mRNA), ribosomal RNA (rRNA), transfer RNA (tRNA), nuclear RNA (nRNA), small interfering RNA (siRNA), small nuclear RNA (snRNA), small nucleolar RNA (snoRNA), small Cajal body-specific RNA (scaRNA), microRNA (miRNA), double stranded (dsRNA), ribozyme, riboswitch or viral RNA, or any nucleic acids that may be obtained from a sample.

In some embodiments, two cells of different kinds (e.g. a first cell and a second cell) can be incubated or cultured to allow for the cells to interact in order to determine a cell-cell interaction or other cell-cell relationship. Interactions between cells can occur via intracellular proteins, extracellular matrix proteins or cell-surface proteins. For example, the first cell can display a receptor molecule capable of binding to a ligand molecule displayed on the second cell. The first cell can be an endothelial cell expressing a selectin and the second cell may be a leukocyte expressing a glycoprotein. In another example, the first cell can be a T cell displaying a T-cell receptor and the second cell can be an antigen presenting cell presenting an antigen. Using the method disclosed herein, the identity of cell-cell interaction and their genomic information can be correlated using the barcode sequencing.

The interaction between the first cell and the second cell can comprise an activating interaction of the first cell by the second cell, an inhibiting interaction of the first cell by the second cell, an inductive interaction of the first cell by the second cell, or a combination thereof. In some embodiments, the activating interaction can comprise an increase in a gene expression level and/or a protein expression level, an increase in cell proliferation, an increase in cell viability, an increase in cell movement, an increase in cell differentiation, an expression of a specific protein or an elevation of its expression level, a secretion of a specific cytokine or elevation of its secretion amount, or a combination thereof. In some embodiments, the inductive interaction can comprise production of proteins or small molecules such as cytokines or chemokines from the first cell and/or the second cell that can confer growth, survival, proliferation, or drug resistance of the first cell and/or the second cell. In some embodiments, the inhibiting interaction can comprise an inhibition of cell movement, a reduction of cell proliferation, a decrease in cell viability, an inhibition in cell differentiation, a decrease in protein expression, a decrease in cytokine secretion, or a combination thereof. For example, a first cell can be a T cell and a second cell can be a B cell, and the interaction between the T cell and the B cell can result in functionalization of the T cell and/or death of the B cell.

In some embodiments, the first cells and the second cells (e.g. cells potentially involved in a cell-cell interaction) are co-partitioned. The plurality of the first cells and the plurality of second cells can be mixed together to form a mixture. The ratio of the first cells and the second cells in a mixture can be different in different embodiments. In some embodiments, the ratio of the first cells and the second cells in a mixture is, is about, is at least, is at least about, is at most, is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the ratio of the first cells and the second cells in a mixture can be 1:1.

In some embodiments, the plurality of cells (e.g. first cells and/or second cells) can be diluted prior to partitioning to ensure majority of the partitions comprise at most one cell with low doublets (more than one cell in one partition). A dilution can be prepared such that a desired cell concentration is achieved. The cell concentration can be between 1×10⁴ and 1×10⁶ (e.g. about, at least, at least about, at most, at most about, 1×10⁴, 2×10⁴, 3×10⁴, 4×10⁴, 5×10⁴, 6×10⁴, 7×10⁴, 8×10⁴, 9×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 1×10⁶, or a number or a range between any two of these values) cells/mL. In some embodiments, the cell concentration is about 1×10⁵-3×10⁵ (e.g. about, at least, at least about, at most, at most about, 1×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2.0×10⁵, 2.1×10⁵, 2.2×10⁵, 2.3×10⁵, 2.4×10⁵, 2.5×10⁵, 2.6×10⁵, 2.7×10⁵, 2.8×10⁵, 2.9×10⁵, 3.0×10⁵, or a number or a range between any two of these values).

Beads

In some embodiments, the plurality of barcode molecules introduced into the plurality of partitions are associated with a bead. The beads can provide a surface upon which molecules, such as oligonucleotides, can be synthesized or attached. In some embodiments, a bead comprises, comprises about, comprises at least, comprises at least about, comprises at most, or comprises at most about 1, 5, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 7000000, 8000000, 9000000, 10000000, 20000000, 50000000, 100000000, 200000000, 300000000, 400000000, 500000000, 600000000, 700000000, 800000000, 900000000, 1000000000, or a number or a range between any two of these values, barcode molecules. FIGS. 1A-1B show each bead attached with a barcode molecule for illustrative purposes and is not intended to be limiting. The attachment can be reversible or irreversible. The attachment can be covalent or non-covalent via non-covalent bonds such as ionic bonds, hydrogen bonds, or van der Waals interactions. The attachment can be direct to the surface of a bead or indirect through other oligonucleotide sequences attached to the surface of a bead.

A bead can be dissolvable, degradable, or disruptable. A bead can be a gel bead such as a hydrogel bead. In some embodiments, the gel bead is degradable upon application of a stimulus. The stimulus can comprise a thermal stimulus, a chemical stimulus, a biological stimulus, a photo-stimulus, or a combination thereof.

A bead can be a solid bead and/or a magnetic bead. In some embodiments, the bead is a magnetic bead. The magnetic bead can comprise a paramagnetic material coated or embedded in the magnetic bead (e.g. on a surface, in an intermediate layer, and/or mixed with other materials of the magnetic bead). A paramagnetic material refers to a material having a magnetic susceptibility slightly greater than 1 (e.g. between about 1 and about 5). A magnetic susceptibility is a measure of how much a material can become magnetized in an applied magnetic field. Paramagnetic materials include, but not limited to, magnesium, molybdenum, lithium, aluminum, nickel, tantalum, titanium, iron oxide, gold, copper, or a combination thereof.

In some embodiments, the magnetic bead comprising barcode molecules can be immobilized or retained in a partition (e.g. a microwell) by an external magnetic field, thereby retaining the barcode molecules in a partition. The magnetic bead comprising barcode molecules can be mobilized or released when the external magnetic field is removed.

In some embodiments, a bead can be immobilized or retained in a partition (e.g. a microwell) through an interaction between two members of a binding pair. For example, the partition (e.g. microwell) can be coated with a capture moiety (e.g. a member of a binding pair) capable of binding with a binding moiety (the other member of the binding pair) comprised in or conjugated to a bead, such that the binding of the two moieties results in the attachment of the bead to the partition (e.g. microwell), thereby immobilizing or retaining the bead in the partition. For example, the surface of a partition (e.g. microwell) can be coated with streptavidin. The biotinylated bead can be attached to the surface of the partition (e.g. microwell) via streptavidin-biotin interaction.

Beads can be of uniform size or heterogeneous size. In some embodiments, the beads have a diameter of about, at least, at least about, at most, or at most about, 1 μm, 5 μm, 10 μm, 20 μm, 30 μm, 40 μm, 45 μm, 50 μm, 60 μm, 65 μm, 70 μm, 75 μm, 80 μm, 90 μm, 100 μm, 250 μm, 500 μm, or 1 mm.

In some embodiments, a bead can be sized such that at most one bead (and a first cell and/or a second cell), not two beads, can fit one partition. A size or dimension (e.g., length, width, depth, radius, or diameter) of a bead can be different in different embodiments. In some embodiments, a size or dimension of one, or each, bead is, is about, is at least, is at least about, is at most, or is at most about, 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, a size or dimension of one, or each, bead is about 1 nm to about 100 μm. As another example, the bead can have a dimension about 10 μm to about 100 μm. As another example, the bead can have a dimension about 30 μm.

The volume of one, or each, bead can be different in different embodiments. The volume of one, or each, bead can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 10000000 nm³, 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm³, or a number or a range between any two of these values. The volume of one, or each, bead can be, be about, be at least, be at least about, be at most, or be at most about, 1 nanolieter (nL), 2 nL, 3 nL, 4 nL, 5 nL, 6 nL, 7 nL, 8 nL, 9 nL, 10 nL, 11 nL, 12 nL, 13 nL, 14 nL, 15 nL, 16 nL, 17 nL, 18 nL, 19 nL, 20 nL, 21 nL, 22 nL, 23 nL, 24 nL, 25 nL, 26 nL, 27 nL, 28 nL, 29 nL, 30 nL, 31 nL, 32 nL, 33 nL, 34 nL, 35 nL, 36 nL, 37 nL, 38 nL, 39 nL, 40 nL, 41 nL, 42 nL, 43 nL, 44 nL, 45 nL, 46 nL, 47 nL, 48 nL, 49 nL, 50 nL, 51 nL, 52 nL, 53 nL, 54 nL, 55 nL, 56 nL, 57 nL, 58 nL, 59 nL, 60 nL, 61 nL, 62 nL, 63 nL, 64 nL, 65 nL, 66 nL, 67 nL, 68 nL, 69 nL, 70 nL, 71 nL, 72 nL, 73 nL, 74 nL, 75 nL, 76 nL, 77 nL, 78 nL, 79 nL, 80 nL, 81 nL, 82 nL, 83 nL, 84 nL, 85 nL, 86 nL, 87 nL, 88 nL, 89 nL, 90 nL, 91 nL, 92 nL, 93 nL, 94 nL, 95 nL, 96 nL, 97 nL, 98 nL, 99 nL, 100 nL, or a number or a range between any two of these values. For example, the volume of one, or each, bead is about 1 nm³ to about 1000000 μm³.

The number of beads introduced into a plurality of partitions can be different in different embodiments. In some embodiments, the number of beads introduced into a plurality of partitions is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number of beads introduced into a plurality of partitions (e.g. microwells) can be at least 80,000 beads.

In some embodiments, beads are introduced to the partitions such that the percentage of partitions each occupied with one bead is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 80% of the plurality of partitions can be each occupied with one bead.

In some embodiments, beads are introduced to the partitions such that the percentage of partitions with no bead is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. For example, at most 20% of the plurality of partitions contain no bead.

### Barcoding Sample Nucleic Acids

The method described herein can comprise barcoding a plurality of sample nucleic acids associated with the first cell and/or the second cell in the partition using the plurality of barcode molecules to generate a plurality of barcoded nucleic acids. FIGS. 1A-1B show barcoding an mRNA molecule with a barcode molecule. The barcode molecule is shown attached to a bead for illustrative purposes and is not intended to be limiting.

Prior to barcoding the sample nucleic acids, the method can comprise lysing cells (e.g. after introducing a plurality of barcode molecules, a plurality of first cells, and/or the plurality of second cells to the partition) to release the content of the first cell and/or the second cell within the partition. Lysis agents can be contacted with the cells or cell suspension concurrently, or immediately after the introduction of the cells into the partition and before the barcoding, e.g. through the flow channels. Examples of lysis agents include bioactive reagents, such as lysis enzymes, or surfactant based lysis solutions including non-ionic surfactants such as TritonX-100 and Tween 20 and ionic surfactants such as sodium dodecyl sulfate (SDS). Lysis methods including, but not limited to, thermal, acoustic, electrical, or mechanical cellular disruption can also be used.

Synthesis of Single-Stranded Barcoded Nucleic Acids

In some embodiments, barcoding a plurality of sample nucleic acids (e.g., mRNA shown in FIGS. 1A-1B) associated with the first cell and/or the second cell in the partition can comprise extending the plurality of barcode molecules using the plurality of sample nucleic acids as templates to generate partially single-stranded/partially double-stranded barcoded nucleic acids of the plurality of barcoded nucleic acids hybridized to sample nucleic acids of the plurality of sample nucleic acids. The partially single-stranded/partially double-stranded barcoded nucleic acids hybridized to sample nucleic acids can be separated by denaturation (e.g., heat denaturation or chemical denaturation using for example, sodium hydroxide) to generate single-stranded barcoded nucleic acids of the plurality of barcoded nucleic acids. The single-stranded barcoded nucleic acids can comprise a barcode molecule and an oligonucleotide complementary to the sample nucleic acids. In some embodiments, the single-stranded barcoded nucleic acids can be generated by reverse transcription using a reverse transcriptase. For example, the single-stranded barcoded nucleic acids can be generated by using a DNA polymerase.

In some embodiments, the single-stranded barcoded nucleic acids can be cDNA produced by extending a barcode molecule using a sample RNA associated with the first cell and/or the second cell as a template. The single-stranded barcoded nucleic acids can be further extended using a template switching oligonucleotide (TSO). A TSO is an oligo that hybridizes to untemplated C nucleotides added by a reverse transcriptase during reverse transcription. The TSO can be introduced into the partitions together with the reverse transcription reagents. For example, a reverse transcriptase can be used to generate a cDNA by extending a barcode molecule hybridized to an RNA. After extending the barcode molecule to the 5′-end of the RNA, the reverse transcriptase can add one or more nucleotides with cytosine (C) bases (e.g. two or three) to the 3′-end of the cDNA. The TSO can include one or more nucleotides with guanine (G) bases (e.g. two or more) on the 3′-end of the TSO. The nucleotides with G bases can be ribonucleotides. The G bases at the 3′-end of the TSO can hybridize to the cytosine bases at the 3′-end of the cDNA. The reverse transcriptase can further extend the cDNA using the TSO as the template to generate a cDNA with the reverse complement of the TSO sequence on its 3′-end. The barcoded nucleic acid can include the barcode sequences (e.g., cell barcode and UMI) on the 5′-end and a TSO sequence at its 3′-end.

In some embodiments, barcoding a plurality of sample nucleic acids comprises extending the barcode molecules using the sample nucleic acids as templates and the plurality of barcode molecules as TSO to generate a plurality of single-stranded barcoded nucleic acids of the plurality of barcoded nucleic acids that are hybridized to the plurality of sample nucleic acids.

In some embodiments, the barcode molecules are not attached to a bead and the barcode molecules can be TSO. For example, extension primers (e.g. poly(dT)) can be introduced into the partitions which hybridize to a sample nucleic acid (e.g. the poly-adenylated mRNA). The extension primers can be extended using the sample nucleic acids as a template. For example, a reverse transcriptase can be used to generate a cDNA by extending an extension primer hybridized to an RNA. After extending the extension primers to the 5′-end of the RNA, the reverse transcriptase can add one or more C bases (e.g. two or three) to the 3′-end of the cDNA. The TSO or barcode molecule can include one or more G bases (e.g. two or more) on the 3′-end of the TSO. The nucleotides with guanine bases can be ribonucleotides. The G bases at the 3′-end of the TSO or barcode molecule can hybridize to the cytosine bases at the 3′-end of the cDNA. The reverse transcriptase can switch template from the mRNA to the TSO or barcode molecule. The reverse transcriptase can further extend the cDNA using the TSO or barcode molecule as the template to generate a cDNA further comprising the reverse complement of the TSO or barcode molecule. In this case, the barcode sequences (e.g. cell barcode and UMI) are on the 3′-end of the generated cDNA.

The single-stranded barcoded nucleic acids can be separated from the template sample nucleic acids by digesting the template sample nucleic acids (e.g., using RNase), by chemical treatment (e.g., using sodium hydroxide), by hydrolyzing the template sample nucleic acids, or via a denaturation or melting process by increasing the temperature, adding organic solvents, or increasing pH. Following the melting process, the sample nucleic acids can be removed (e.g. washed away) and the single-stranded barcoded nucleic acids can be retained in the partition (e.g. through attachment to the partitions or through attachments to beads which can be retained in the partitions).

Synthesis of Double-Stranded Barcoded Nucleic Acids

In some embodiments, barcoding a plurality of sample nucleic acids associated with the first cell and/or the second cell in the partition can comprise generating the plurality of barcoded nucleic acids comprising double-stranded barcoded nucleic acids in the partition using the single-stranded barcoded nucleic acids as templates. The double-stranded barcoded nucleic acids can be generated from the single-stranded barcoded nucleic acids retained in the partition using, for example, second-strand synthesis or one-cycle PCR (see Example 1 for an example).

The generated double-stranded barcoded nucleic acid can be denaturized or melted to generate two single-stranded barcoded nucleic acids: one single-stranded barcoded nucleic acid retained in the partition (e.g., attached to the bead) and the other single-stranded barcoded nucleic acid released into the solution from the retained single-stranded barcoded nucleic acid that can then be pooled to provide a pooled mixture outside the partitions. Both single-stranded barcoded nucleic acids (e.g. retained in the partitions or pooled outside the partitions) have a sequence comprising a sequence of a barcode molecule (e.g. a cell barcode sequence and/or a UMI barcode) and a sequence of a sample nucleic acid or a reverse complement thereof.

Barcodes

The term “barcode” as used herein generally can be a verb or a noun. When used as a noun, the term “barcode” or “barcode molecule” refers to a label that can be attached to a polynucleotide, or any variant thereof, to convey information about the polynucleotide. For example, a barcode can be a polynucleotide sequence attached to all fragments of the sample nucleic acids associated with the first cell and/or the second cell in the partition. The barcode can then be sequenced alone or with the fragments of the sample nucleic acids associated with the first cell and/or the second cell. The presence of the same barcode on multiple sequences or different barcodes on different sequences can provide information about the cell origin and/or the molecular origin of the sequences. When used as a verb, the term “barcode” refers to a process of attaching a barcode or a barcode molecule to a sample nucleic acid associated with the first cell and/or the second cell. The barcode molecules can be attached to a partition directly or indirectly. The barcode molecules can also be associated with beads.

Barcode molecules can be generated from a variety of different formats, including pre-designed polynucleotide barcodes, randomly synthesized barcode sequences, microarray-based barcode synthesis, random N-mers, or combinations thereof as will be understood by a person skilled in the art.

In some embodiments, the plurality of barcode molecules comprise, comprise about, comprise at least, comprise at least about, comprise at most, or comprise at most about 1, 5, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 7000000, 8000000, 9000000, 10000000, 20000000, 50000000, 100000000, 200000000, 300000000, 400000000, 500000000, 600000000, 700000000, 800000000, 900000000, 1000000000, or a number or a range between any two of these values.

A barcode molecule of the plurality of barcode molecules can be in any suitable length. In some embodiments, a barcode molecule of the plurality of barcode molecules can be about 2 to about 500 nucleotides in length, about 2 to about 100 nucleotides in length, about 2 to about 50 nucleotides in length, about 2 to about 40 nucleotides in length, about 4 to about 20 nucleotides in length, or about 6 to 16 nucleotides in length.

In some embodiments, a barcode molecule of the plurality of barcode molecules is about, at least, at least about, at most, or at most about 2, 3, 4, 5, 6, 7, 8, 9, 10, 11, 12, 13, 14, 15, 16, 17, 18, 19, 20, 25, 30, 35, 40, 45, 50, 55, 60, 65, 70, 85, 90, 95, 100, 150, 200, 250, 300, 400, or 500 nucleotides in length, or a number or a range between any two of these values.

Each of the plurality of barcode molecules used herein can comprise a cell barcode (e.g. a first barcode sequence) and a molecular barcode (e.g. a second barcode sequence or UMI) (see FIGS. 1A-1B). A barcode molecule can also comprise a target binding sequence or region capable of hybridizing to sample nucleic acids (e.g. poly(dT) sequence in FIGS. 1A-1B). A barcode molecule can also include additional sequence segments such as additional recognition or binding sequences, a template switching oligonucleotide, and primer sequences (e.g. sequencing primer sequence, such as Read 1, in FIG. 1A or a PCR primer sequence in FIG. 1B) for subsequent processing (e.g. PCR amplification) and/or sequencing.

The configuration of the various sequences comprised in a barcode molecule of the plurality of barcode molecules introduced into a partition (e.g. cell barcode sequence, UMI, primer sequence, target binding sequence or region, and/or any additional sequences) can vary depending on, for example, the particular configuration desired and/or the order in which the various components of the sequence are added as will be understood to a person skilled in the art. In some embodiments, a barcode molecule has a configuration of 5′-primer sequence-cell barcode-UMI-target binding sequence-3′. In some embodiments, a barcode molecule has a configuration of 5′-primer sequence-cell barcode-UMI-template switching oligonucleotide-3′.

Cell Barcode

In some embodiments, the first barcode sequences of the barcode molecules are cell barcodes for identifying the plurality of barcoded nucleic acids originate from the first cell and/or the second cell. The cell barcodes of the barcode molecules in a partition can be identical or different.

In some embodiments, the cell barcodes can serve to track the sample nucleic acids associated with the first cell and/or the second cell throughout the processing (e.g., location of the cells in the plurality of partitions) when the cell barcode associated with the sample nucleic acids is read during sequencing. In some embodiment, the cell barcodes can serve to provide linkage information between cell nucleic acid sequences and cell functionality when in combination with optical imaging. Optical imaging can be performed to identify a partition with an interaction between the first cell (e.g., a T cell) and the second cell (e.g., a B cell) that is of interest. Barcoded nucleic acids with an identical cell barcode can be generated from sample nucleic acids of interacting cells within a given partition. Some barcoded nucleic acids are pooled and sequenced to determine cell nucleic acid sequences or a profile (e.g., an mRNA expression profile) which is associated with (e.g., identifiable by or linked with) the cell barcode sequence. The cell barcode sequence of the barcoded nucleic acids that remain or are retained in the partition can be determined using, for example, fluorescent probes by sequencing by ligation. The partition can be identified with the cell barcode sequence. By matching the cell barcode sequence associated with the profile and the cell barcode sequence of the partition, the profile can be tracked to the first cell and/or the second cell and linked to the interaction of interest. For example, the profile can be tracked to that partition where phenotypic observables (e.g. cellular metabolism, cell cycle states, cell signaling, cell viability etc.) of the cells have been obtained or recorded. The profile can thus be linked to the observed cell-cell interaction.

The number (or percentage) of barcode molecules introduced in a partition with cell barcodes having an identical sequence can be different in different embodiments. In some embodiments, the number of barcode molecules introduced in a partition with cell barcodes having an identical sequence is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode molecules introduced in a partition with cell barcodes having an identical sequence is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the cell barcodes of at least two barcode molecules introduced in a partition comprise an identical sequence.

A cell barcode can be unique (or substantially unique) to a partition. The number of unique cell barcode sequences can be different in different embodiments. In some embodiments, the number of unique cell barcode sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of unique cell barcode sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 8100, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values, of the cell barcode sequences of the barcode molecules introduced in a partition. For example, the cell barcodes of barcode molecules introduced in two partition can comprise different sequences.

In some embodiments, barcode molecules are introduced to the plurality of partitions such that different sets of a plurality of barcode molecules introduced in different partitions have different cell barcode and a same set of plurality of barcode molecules introduced in a same partition have same cell barcode. For example, the first cell and/or the second cell partitioned in a same partition of the plurality of partitions will be barcoded with the same cell barcode.

The length of a cell barcode of a barcode molecule (or a cell barcode of each barcode molecule or all cell barcodes of the plurality of barcode molecules) can be different in different embodiments. In some embodiments, a cell barcode of a barcode molecule (or each cell barcode of each barcode molecule or all cell barcodes of the plurality of barcode molecules) is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

In some embodiments, a cell barcode of a barcode molecule (or each cell barcode of each barcode molecule or all cell barcodes of the plurality of barcode molecules) has a length greater than 2 nucleic acid bases. In some embodiments, a cell barcode of a barcode molecule (or each cell barcode of each barcode molecule or all cell barcodes of the plurality of barcode molecules) is 2-40 nucleotides in length. In some embodiments, a cell barcode of a barcode molecule (or each cell barcode of each barcode molecule or all cell barcodes of the plurality of barcode molecules) is at least 6 nucleic acid bases in length.

UMI

In some embodiments, the second barcode sequences of the barcode molecules are unique molecule identifiers (UMIs) for identifying molecular origins of the plurality of barcoded nucleic acids. UMIs are short sequences used to uniquely tag each molecule in a sample in some embodiments. The UMIs of the barcode molecules of the plurality of barcode molecules partitioned into a partition can be identical or different.

In some embodiments, the UMIs of the plurality of barcode molecules are different. The number (or percentage) of UMIs of barcode molecules introduced in a partition with different sequences can be different in different embodiments. In some embodiments, the number of UMIs of barcode molecules introduced in a partition with different sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of UMIs of barcode molecules introduced in a partition with different sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the UMIs of two barcode molecules of the plurality of barcode molecules introduced in a partition can comprise different sequences.

The number of barcode molecules introduced in a partition with UMIs having an identical sequence can be different in different embodiments. In some embodiments, the number of barcode molecules introduced in a partition with UMIs having an identical sequence is, is about, is at least, is at least about, is at most, or is at most about, 1, 2, 3, 4, 5, 6, 7, 8, 9, 10, 20, 30, 40, 50, 60, 70, 80, 90, 100, or a number or a range between any two of these values. For example, the UMIs of two barcode molecules introduced in a partition can comprise an identical sequence.

The number of unique UMI sequences can be different in different embodiments. In some embodiments, the number of unique UMI sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

The length of a UMI of a barcode molecule (or a UMI of each barcode molecule) can be different in different embodiments. In some embodiments, a UMI of a barcode molecule (or a UMI of each barcode molecule) is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

In some embodiments, the UMIs have a length greater than 2 nucleic acid bases. In some embodiments, the UMIs are 2-40 nucleotides in length. In some embodiments, the UMIs are at least 6 nucleic acid bases in length.

Primer Sequence

In some embodiments, a barcode molecule (or each barcode molecule of the plurality of barcode molecules) can comprise a primer sequence. The primer sequence can be a sequencing primer sequence (or a sequencing primer binding sequence) as illustrated in FIG. 1A or a PCR primer sequence (or PCR primer binding sequence) as illustrated in FIG. 1B. For example, the sequencing primer is a Read 1 sequence.

Target Binding Sequence

In some embodiments, a barcode molecule (or each barcode molecule of the plurality of barcode molecules) can comprise a target binding sequence or region capable of hybridizing to a plurality of sample nucleic acids, a particular type of sample nucleic acids (e.g. mRNA), and/or specific sample nucleic acids (e.g. specific gene of interest).

The length of a target binding sequence can be different in different embodiments. In some embodiments, a target binding sequence is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. The target binding sequence can be 12-18 deoxythymidines in length. In some embodiments, the target binding sequence can be 20 nucleotides or longer to enable their annealing in reverse transcription reactions at higher temperatures as will be understood by a person skilled in the art.

In some embodiments, barcode molecules comprising target binding sequences can be introduced into the partitions together with other reagents such as the reverse transcription reagents. The number of the barcode molecules introduced into a partition comprising a target binding sequence can be different in different embodiments. In some embodiments, the number of barcode molecules introduced into a partition comprising a target binding sequence (e.g., poly(dT) sequence) is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

In some embodiments, the target binding sequence can be on a 3′ end of a barcode molecule of the plurality of barcode molecules introduced in a partition. Barcode molecules each comprising a poly(dT) target binding sequence can be used to capture (e.g., hybridize to) 3′ end of polyadenylated mRNA transcripts in a sample nucleic acid for a downstream 3′ gene expression library construction.

In some embodiments, the target binding sequence can comprise a poly(dT) sequence which is a single-stranded sequence of deoxythymidine (dT) used for first-strand cDNA synthesis catalyzed by reverse transcriptase. In some embodiments, the target binding sequence comprises a poly(dT) sequence can be introduced into the partitions as extension primers to synthesize the first-strand cDNA using the sample nucleic acid (e.g. RNA) as a template.

In some embodiments, the poly(dT) of the barcode molecules introduced into a partition can be identical (e.g., same number of dTs). In some embodiments, the poly(dT) of the barcode molecules introduced into a partition can be different (e.g. different numbers of dTs). The percentage of the barcode molecules of the plurality of barcode molecules introduced into a partition with an identical poly(dT) sequence can be different in different embodiments. In some embodiments, the percentage of the barcode molecules of the plurality of barcode molecules introduced into a partition with an identical poly(dT) sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

In some embodiments, the target binding regions of all barcode molecules of the plurality of barcode molecules comprise poly(dT) capable of hybridizing to poly(A) tails of mRNA molecules (or poly(dA) regions or tails of DNA). In some embodiments, the target binding regions of some barcode molecules of the plurality of barcode molecules comprise gene-specific or target-specific primer sequences. For example, a barcode molecule of the plurality of barcode molecules can also comprise a target binding region capable of hybridizing to a specific sample nucleic acid associated with the first cell and/or second cell, thereby capturing specific targets or analytes of interest. For example, the target binding region capable of hybridizing to a specific sample nucleic acid can be a gene-specific primer sequence. The gene-specific primer sequences can be designed based on known sequences of a target nucleic acid of interest. The gene-specific primer sequences can span a nucleic acid region of interest, or adjacent (upstream or downstream) of a nucleic acid region of interest.

The length of a gene-specific primer sequence can be different in different embodiments. In some embodiments, a gene-specific primer sequence is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. For example, a gene-specific primer sequence is at least 10 nucleotides in length.

The number of the barcode molecules introduced into a partition comprising a gene-specific primer sequence can be different in different embodiments. In some embodiments, the number of barcode molecules introduced into a partition comprising a gene-specific primer sequence is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

In some embodiments, the barcode molecules introduced into a partition can comprise a set of different gene-specific primer sequences each capable of binding to a specific target nucleic acid sequence.

The number of different gene-specific primer sequences of the barcode molecules introduced into a partition can be different in different embodiments. In some embodiments, the number of different gene-specific primer sequences of the barcode molecules introduced into a partition is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values.

Accordingly, the number of target nucleic acids of interest (e.g. genes of interest) that the barcode molecules introduced into a partition are capable of binding can be different in different embodiments. In some embodiments, the number of target nucleic acids of interest (e.g. genes of interest) the barcode molecules introduced into a partition are capable of binding is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values. One barcode molecule introduced into a partition can bind to a molecule (or a copy) of a target nucleic acid. Barcode molecules introduced into a partition can bind to molecules (or copies) of a target nucleic acid or a plurality of target nucleic acids.

In some embodiments, the barcode molecules of the plurality of barcode molecules can each comprise a poly(dT) sequence, a gene-specific primer sequence, and/or both. The poly(dT) sequence and the gene-specific primer sequence can be on a same barcode molecule or different barcode molecules of the plurality of barcode molecules introduced into a partition.

The ratio of the number of barcode molecules introduced into a partition comprising a poly(dT) sequence and the number of barcode molecules introduced into a partition comprising a gene-specific primer sequence can be different in different embodiments. In some embodiments, the ratio is, is about, is at least, is at least about, is at most, is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values.

Template Switching Oligonucleotide

In some embodiments, a barcode molecule (or each barcode molecule of the plurality of barcode molecules) can be a template switching oligonucleotide. A primer comprising a target binding region, such as a poly(dT) sequence, can hybridize to a sample nucleic acid (e.g., an mRNA) and be extended by, for example, reverse transcription to generate an extended primer comprising a reverse complement of the sample nucleic acid, or a portion thereof (e.g., cDNA). The extended primer or cDNA can be further extended to include the reverse complement of a TSO oligonucleotide or barcode molecule as illustrated in FIGS. 1A-1B. The resulting barcoded nucleic acid includes the barcodes of the barcode molecule on the 3′-end. In some embodiments, a barcode molecule (or each barcode molecule of the plurality of barcode molecules) is not a template switching oligonucleotide. A barcode molecule comprising a target binding region, such as a poly(dT) sequence, can hybridize to a sample nucleic acid (e.g., an mRNA) and be extended by, for example, reverse transcription to generate an extended primer comprising a reverse complement of the sample nucleic acid, or a portion thereof (e.g., cDNA). The extended primer or cDNA can be further extended to include the reverse complement of a TSO oligonucleotide. The resulting barcoded nucleic acid includes the barcodes of the barcode molecule on the 5′-end.

A template switching oligonucleotide (TSO) is an oligonucleotide that hybridizes to untemplated C nucleotides added by a reverse transcriptase during reverse transcription. The TSO can hybridize to the 3′ end of a cDNA molecule. The TSO can include one or more nucleotides with guanine (G) bases on the 3′-end of the TSO, with which the one or more cytosine (C) bases added by a reverse transcriptase to the 3′-end of a cDNA can hybridize. The series of G bases can comprise IG base, 2 G bases, 3 G bases, 4 G bases, 5 G bases or more than 5 G bases. The series of G bases can be ribonucleotides. The reverse transcriptase can further extend the cDNA using the TSO as the template to generate a barcoded cDNA comprising the TSO.

The length of a TSO can be different in different embodiments. In some embodiments, a template switching oligonucleotide is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

In some embodiments, the TSO can have a length greater than 2 nucleic acid bases. In some embodiments, the template switching oligonucleotides are 2-40 nucleotides in length. In some embodiments, the template switching oligonucleotides are at least 12 nucleic acid bases in length.

The number of the barcode molecules introduced into a partition comprising a TSO can be different in different embodiments. In some embodiments, the number of barcode molecules introduced into a partition comprising a TSO is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

In some embodiments, the TSO of the barcode molecules introduced into a partition can be identical. In some embodiments, the TSO of the barcode molecules introduced into a partition can be different. The percentage of the barcode molecules of the plurality of barcode molecules introduced into a partition with an identical TSO sequence can be different in different embodiments. In some embodiments, the percentage of the barcode molecules of the plurality of barcode molecules introduced into a partition with an identical TSO sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

### Pooling

In some embodiments, the method comprises pooling barcoded nucleic acids of the plurality of barcoded nucleic acids after barcoding the sample nucleic acids and before sequencing the barcoded nucleic acids to obtain pooled barcoded nucleic acids.

In some embodiments, pooling barcoded nucleic acids occurs after generating the double-stranded barcoded nucleic acids. In some embodiments, pooling barcoded nucleic acids occurs after denaturizing (such as heat denaturization or chemical denaturization with, for example, sodium hydroxide) the double-stranded barcoded nucleic acids which generates two single-stranded barcoded nucleic acids, one retained in the partition and one released from the barcoded nucleic acids retained in the partition. In some embodiments, pooling barcoded nucleic acids comprises collecting the single-stranded barcoded nucleic acids released from the barcoded nucleic acids retained in the partition.

In some embodiments wherein the barcode molecules are attached to beads, only single-stranded barcoded nucleic acids released into bulk are collected by pooling, and the beads are not pooled (e.g. not removed from the partitions) but retained in the partitions (e.g. by an external magnetic field applied on magnetic beads), thereby allowing one to trace the origin of the pooled barcoded nucleic acids, for example, to its original location in the plurality of partitions.

The pooled barcoded nucleic acids can be single-stranded or double-stranded (e.g. generated from the single-stranded pooled barcoded nucleic acids by PCR amplification). The pooled barcoded nucleic acids (e.g. barcoded cDNA) can be purified and/or amplified prior to sequencing library construction. The pooled barcoded nucleic acids with desired length may be selected.

### Sequencing Library Construction

Referring to FIGS. 1A-1B, the barcoded nucleic acids (e.g. pooled barcoded nucleic acids) are further processed prior to sequencing to generate processed barcoded nucleic acids. For example, the method can include amplification of barcoded nucleic acids, fragmentation of amplified barcoded nucleic acids, end repair of fragmented barcoded nucleic acids, A-tailing of fragmented barcoded nucleic acids that have been end-repaired (e.g., to facilitate ligation to adapters), and attaching (e.g. by ligation and/or PCR) with a second sequencing primer sequence (e.g. a Read 2 sequence), sample indexes (e.g. short sequences specific to a given sample library), and/or flow cell binding sequences (e.g. P5 and/or P7). Additional PCR amplification can also be performed. This process can also be referred to as sequencing library construction.

In some embodiments, the method comprises performing a polymerase chain reaction in bulk, subsequent to the pooling, on the pooled barcoded nucleic acids, thereby generating amplified barcoded nucleic acids. PCR amplification can be carried out to generate sufficient mass for the subsequent library construction processes. PCR amplification can also be performed with primers specific to target nucleic acids of interest such as T-cell receptor (TCR) or B-cell receptor (BCR) constant regions.

In some embodiments, the method comprises fragmenting (e.g., via enzymatic fragmentation, mechanical force, chemical treatment, etc.) the pooled barcoded nucleic acids to generate fragmented barcoded nucleic acids. Fragmentation can be carried out by any suitable process such as physical fragmentation, enzymatic fragmentation, or a combination of both. For example, the barcoded nucleic acids can be sheared physically using acoustics, nebulization, centrifugal force, needles, or hydrodynamics. The barcoded nucleic acids can also be fragmented using enzymes, such as restriction enzymes and endonucleases.

Fragmentation yields fragments of a desired size for subsequent sequencing. The desired sizes of the fragmented nucleic acids are determined by the limitations of the next generation sequencing instrumentation and by the specific sequencing application as will be understood by a person skilled in the art. For example, when using Illumina technology, the fragmented nucleic acids can have a length of between about 50 bases to about 1,500 bases. In some embodiments, the fragmented barcoded nucleic acids have about 100 bp to 700 bp in length.

Fragmented barcoded nucleic acids can undergo end-repair and A-tailing (to add one or more adenine bases) to form an A overhang. This A overhang allows adapter containing one or more thymine overhanging bases to base pair with the fragmented barcoded nucleic acids.

Fragmented barcoded nucleic acids can be further processed by adding additional sequences (e.g. adapters) for use in sequencing based on specific sequencing platforms. Adapters can be attached to the fragmented barcoded nucleic acids by ligation using a ligase and/or PCR. For example, fragmented barcoded nucleic acids can be processed by adding a second sequencing primer sequence. The second sequencing primer sequence can comprise a Read 2 sequence. An adapter comprising the second primer sequence can be ligated to the fragmented barcoded nucleic acids after, for example, end-repair and A tailing, using a ligase. The adaptor can include one or more thymine (T) bases that can hybridize to the one or more A bases added by A tailing. An adaptor can be, for example, partially double-stranded or double stranded.

The adapter can also include platform-specific sequences for fragment recognition by specific sequencing instrument. For example, the adapter can comprise a sequence for attaching the fragmented barcoded nucleic acids to a flow well of Illumina platforms, such as a P5 sequence, a P7 sequence, or a portion thereof. Different adapter sequences can be used for different next generation sequencing instrument as will be understood by a person skilled in the art.

The adapter can also contain sample indexes to identify samples and to permit multiplexing. Sample indexes enable multiple samples to be sequenced together (i.e. multiplexed) on the same instrument flow cell as will be understood by a person skilled in the art. Adapters can comprise a single sample index or a dual sample indexes depending on the implementations such as the number of libraries combined and the level of accuracy desired.

In some embodiments, the amplified barcoded nucleic acids generated from sequencing library construction can include a P5 sequence, a sample index, a Read 1 sequence, a cell barcode, a UMI, a poly(dT) sequence, a target biding region, a sequence of a sample nucleic acid or a portion thereof, a Read 2 sequence, a sample index, and/or a P7 sequence (e.g., from 5′-end to 3′-end). In some embodiments, the amplified barcoded nucleic acids can include a P5 sequence, a sample index, a Read 1 sequence, a cell barcode, a UMI, a sequence of a template switching oligonucleotide, a sequence of a sample nucleic acid or a portion thereof, a Read 2 sequence, a sample index, and/or a P7 sequence (e.g., from 5′-end to 3′-end).

In some embodiments, sequencing the barcoded nucleic acids, or products thereof, comprises sequencing products of the barcoded nucleic acids. Products of the barcoded nucleic acids can include the processed nucleic acids generated by any step of the sequencing library construction process, such as amplified barcoded nucleic acids, fragmented barcoded nucleic acids, fragmented barcoded nucleic acids comprising additional sequences such as the second sequencing primer sequence and/or adapter sequences described herein.

### Sequencing Barcoded Nucleic Acids

The method disclosed herein can comprise sequencing the plurality of barcoded nucleic acids or products thereof to obtain nucleic acid sequences of the plurality of barcoded nucleic acids. The barcoded nucleic acids generated by the method disclosed herein comprise barcoded nucleic acids retained in a partition and barcoded nucleic acids pooled, from each partition, into a pooled mixture outside the partitions. The barcoded nucleic acids retained in a partition and the pooled barcoded nucleic acids in a pooled mixture outside the partitions can be sequenced using a same or different sequencing techniques.

Sequencing Pooled Barcoded Nucleic Acids

In some embodiments, sequencing the plurality of barcoded nucleic acids or products thereof comprises sequencing the pooled barcoded nucleic acids to obtain nucleic acid sequences of the pooled barcoded nucleic acids. As used herein, a “sequence” can refer to the sequence, a complementary sequence thereof (e.g., a reverse, a compliment, or a reverse complement), the full-length sequence, a subsequence, or a combination thereof. The nucleic acids sequences of the pooled barcoded nucleic acids can each comprise a sequence of a barcode molecule (e.g. the cell barcode and the UMI) and a sequence of a sample nucleic acid associated with the first cell and/or the second cell or a reverse complement thereof.

Pooled barcoded nucleic acids can be sequenced using any suitable sequencing method identifiable to a person skilled in the art. For example, sequencing the pooled barcoded nucleic acids can be performed using high-throughput sequencing, pyrosequencing, sequencing-by-synthesis, single-molecule sequencing, nanopore sequencing, sequencing-by-ligation, sequencing-by-hybridization, next generation sequencing, massively-parallel sequencing, primer walking, and any other sequencing methods known in the art and suitable for sequencing the barcoded nucleic acids generated using the methods herein described.

Sequencing Barcoded Nucleic Acids Retained in the Partitions

In some embodiments, sequencing the plurality of barcoded nucleic acids or products thereof comprises sequencing the barcoded nucleic acids retained in the partitions to obtain the nucleic acid sequences of the retained barcoded nucleic acids. Sequencing the barcoded nucleic acids retained in the partitions can comprise sequencing the entire sequence of a barcoded nucleic acid or sequencing a portion of the sequence of a barcoded nucleic acid, such as the cell barcode sequence of a barcoded nucleic acid. In some embodiments, sequencing the barcoded nucleic acids retained in the partition can comprise determining the cell barcode sequences of the barcoded nucleic acids retained in the partition using oligonucleotide probes each comprising a fluorescent label.

In some embodiments, the cell barcode sequences of the barcoded nucleic acids retained in the partition can be determined using sequencing-by-ligation. The sequencing-by-ligation process can be carried out in the same microfluidic device used for performing other steps of the methods described herein, such as partitioning cells and barcode molecules and barcoding sample nucleic acids, without the necessity to transfer the barcoded nucleic acids elsewhere and therefore can be referred to as on-chip sequencing (see Example 1 for an example).

In the sequencing-by-ligation process, a first sequencing primer is hybridized to a single-stranded barcoded nucleic acid to be sequenced. A mixture (e.g., 16) of n-mer probes (e.g. 8-mer probes) carrying m (e.g., four) distinct fluorescent labels compete for ligation to the first barcode (e.g., cell barcode) right after the first sequencing primer. The number of n-mer probes can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more. The n-mer probes can be, for example, 2-, 3-, 4-, 5-, 6-, 7-, 8-, 9-, 10-, 11-, 12-, 13-, 14-, 15-, 16-, 17-, 18-, 19-, 20-, or more. The number of fluorescent labels used can be, for example, 1, 2, 3, 4, 5, 6, 7, 8, 9, 10, or more. The fluorophore encoding, which is based on the two 3′-most nucleotides of a probe, is read. Three bases including the dye are cleaved from the 5′ end of the probe, leaving a free 5′ phosphate on the extended primer, which is then available for further ligation. After multiple ligations (e.g. 3 rounds of ligation), the synthesized strands are melted and the ligation product is washed away before a second sequencing primer is annealed. A second sequencing primer then hybridizes the single-stranded barcoded nucleic acid at a base position shifted by one nucleotide with respect to the position the first sequencing primer binds to. The ligation process is then repeated for the second sequencing primer. The same process is followed for the rest of the sequencing primers. The dye read outs can be converted to a sequence. In some embodiments, 5 different sequencing primers are provided to sequence the first barcode sequences of the single-stranded barcoded nucleic acids retained in the partition.

In some embodiments, determining the cell barcode sequences of the barcoded nucleic acids retained in the partition using sequencing-by-ligation can comprise introducing a sequencing primer capable of hybridizing to the barcoded nucleic acids retained in the partition. The method can also comprise extending the sequencing primer using the barcoded nucleic acids retained in the partition as templates to generate a plurality of extended sequencing primers comprising the first barcode sequences, or a portion thereof, of the barcoded nucleic acids retained in the partition. For example, a different sequencing primer can be introduced and extended in each of one or more cycles herein described (e.g. one or more cycles of introducing a sequencing primer capable of hybridizing to the barcoded nucleic acids retained in the partition and extending the sequencing primer using the barcoded nucleic acids retained in the partition as templates to generate a plurality of extended sequencing primers comprising the first barcode sequences, or a portion thereof, of the barcoded nucleic acids retained in the partition). The introducing and extending are repeated with a different sequencing primer capable of hybridizing to the barcoded nucleic acids retained in the partition. The method can also comprise introducing a plurality of oligonucleotide probes each comprising a fluorescent label. For example, the plurality of oligonucleotide probes are octamer probes.

### Post-Sequencing Analysis

The obtained nucleic acid sequences of the plurality of barcoded nucleic acids (e.g. nucleic acid sequences of pooled barcoded nucleic acids) can be subjected to any downstream post-sequencing data analysis as will be understood by a person skilled in the art. The sequence data can undergo a quality control process to remove adapter sequences, low-quality reads, uncalled bases, and/or to filter out contaminants. The high-quality data obtained from the quality control can be mapped or aligned to a reference genome or assembled de novo.

Gene expression quantification and differential expression analysis can be carried out to identify genes whose expression differs under different conditions, such as, external stimuli and/or signals received from other cells through cell-cell interaction.

In some embodiments, the method can comprise determining a profile (e.g. an expression profile, an omics profile, or a multi-omics profile) of the sample nucleic acids associated with the first cell and/or the second cell. A profile can be a single omics profile, such as a transcriptome profile. The profile can be a multi-omics profile, which can include profiles of genome (e.g. a genomics profile), proteome (e.g. a proteomics profile), transcriptome (e.g. a transcriptomics profile), epigenome (e.g. an epigenomics profile), metabolome (e.g. a metabolomics profile), and/or microbiome (e.g. microbiome profile). The profile can include an RNA expression profile. The profile can include a protein expression profile. The expression profile can comprise an RNA expression profile, an mRNA expression profile, and/or a protein expression profile. The profile can be a profile of one cell (e.g. a first cell or a second cell). The profiles can be profiles of two cells from a same partition (e.g. a first cell and a second cell). A profile can also be a profile of one or more target nucleic acids (e.g. gene markers) or a selection of genes associated with the first cell and/or the second cell.

In some embodiments, the method disclosed herein can be used to determine a profile (e.g., an expression profile, an omics profile, or a multi-omics profile) of a cell involved in cell-cell interaction, such as to detect changes in gene expression profile of the cell in terms of identification of RNA transcripts and their quantitation. In some embodiments, a profile of a first cell and/or a second cell can be determined using the nucleic acid sequences of the plurality barcoded nucleic acids. For example, determining the profile of the first cell and/or the second cell can comprise determining the profile of the first cell and/or the second cell using the second barcode sequences (e.g. UMI) and sequences of the sample nucleic acids, or a portion thereof, present in the nucleic acid sequences.

In some embodiments, the first cell and the second cell when in contact (or under incubation) with each other can have an expression profile different from an expression profile of the first cell or the second cell alone. A differential expression analysis can be performed to detect quantitative changes in expression levels between the cell involved in a cell-cell interaction and the cell alone. Genes expressed differentially can be detected. Differential expression profile can be correlated to the cell functionality and/or cell's phenotypes.

Therefore, in some embodiments, an interaction between the first cell and the second cell may be of interest. The interaction between the first cell and the second cell that is of interest can comprise a change in a profile of the first cell and/or the second cell. The profile can comprise a transcriptomics profile, a multi-omics profile such as a genomics profile, a proteomics profile, a transcriptomics profile, an epigenomics profile, a metabolomics profile, a chromatics profile, or a combination thereof. In some embodiments, the profile of the first cell and/or the second cell in the partition can be different from a profile of the first cell or the second cell alone.

Correlating Sequence Information with Cell-Cell Interaction

Matching Barcode Sequences

The obtained nucleic acid sequences of barcoded nucleic acids can comprise nucleic acid sequences of pooled barcoded nucleic acids and nucleic acid sequences of retained barcoded nucleic acids. For example, two barcoded nucleic acids of the plurality of barcoded nucleic acids can comprise a retained barcoded nucleic acid of the retained barcoded nucleic acids and a pooled barcoded nucleic acid of the pooled barcoded nucleic acids. The cell barcode sequence of the retained barcoded nucleic acid and the cell barcode sequence of the pooled barcoded nucleic acid can be identical. The barcode sequence (e.g. cell barcode sequence) of the pooled barcoded nucleic acids can be matched to the barcode sequence (e.g. cell barcode sequence) of the retained barcoded nucleic acids. By matching the two cell barcode sequences, the nucleic acid sequences of the pooled barcoded nucleic acids associated with the first cell and/or the second cell can be traced back to the location of the partition the barcoded nucleic acids are pooled from (or the partition the first cell and/or the second cell is originally distributed into), which is the partition where the sample nucleic acids are being barcoded.

In some embodiments, the method can comprise recording a location of the partition comprising the first cell and/or the second cell, or characteristics of the first cell and/or the second cell in the partition to provide a recorded location of the partition, or characteristics of the first cell and/or the second cell in the partition. Recording the location of a partition can be performed subsequent to partitioning a plurality of first cells and/or a plurality of second cells. Recording the location of a partition can be performed prior to barcoding the sample nucleic acids. Recording the location of the partition comprising a first cell and a second cell, or characteristics of the first cell and the second cell therein can comprise optically imaging the partition, or the first cell and the second cell therein. The location of the partition can be a spatial location of the partition within the plurality of partitions, such as a row and column address within a two-dimensional microwell array.

In some embodiments, the method can include matching a recorded location of the partition with the nucleic acid sequences of the pooled barcoded nucleic acids. The recorded location of the partition comprising a first cell and a second cell and/or the characteristic of the first cell and/or the second cell in the partition can be linked with the first barcode sequence (cell barcode sequence) of the barcoded nucleic acids retained in the partition. The first barcode sequence (cell barcode sequence) of the barcoded nucleic acids retained in the partition can then be linked to the first barcode sequence (cell barcode sequence) of the pooled barcoded nucleic acids originating from sample nucleic acids associated with the first cell and/or the second cell.

In some embodiments, matching a recorded location of the partition with the cell barcode sequence of the retained barcoded nucleic acids and then with the nucleic acid sequences (e.g. cell barcode sequence and sample nucleic acid sequences) of the pooled barcoded nucleic acids can comprise the use of a barcode-to-location look-up table, which stores, in rows and columns, a recorded location of a partition (or a partition of interest) within the plurality of partitions and their corresponding barcode sequences. The look-up table can be generated by associating the cell barcode sequence of the barcoded nucleic acids retained in a partition with a recorded location of the partition within the plurality of partitions, such as the location of a microwell in a microwell array. For example, each recorded partition is represented in the look-up table by a pair of values, one value for each of the two columns: “a recorded row/column address” of a microwell of interest within a two-dimensional microwell array and “a cell barcode sequence” introduced into that microwell.

Matching Sequences with Cell-Cell Interaction

In some embodiments, the method can comprise recording a location of the partition comprising a first cell and a second cell, wherein the first cell and second cell can potentially interact with each other thereby exhibiting phenotypic observable features of interest. In some embodiments, prior to recording a location of the partition, the cells can be cultured for a certain time under a condition to allow the first cell and the second cell to interact.

In some embodiments, the method can include recording a location of the partition comprising a first cell and a second cell. The method can comprise recording characteristics of the first cell and/or the second cell in the partition. The characteristics can comprise phenotypic features of the first cell and the second cell in the partition. The phenotypic features of a pair of interacting cells can be used to interpret the nature of the cell-cell interaction.

Phenotypic features obtained for the cells in each partition can include any phenotypic observables such as cellular metabolism, cell cycle states, cell signaling, cell viability, a cell size, a nucleus size, cell morphology or nucleus information, a surface marker expression level, protein expression level (e.g., fluorescence protein expression level), cytokine expression profile, other related signaling dynamics and behavior. Phenotypic features can be obtained using any suitable approach including cell imaging, immunofluorescence, and protein secretion assays. For example, phenotypic features can be obtained using a fluorescence microscopy, a fluorescence imaging, phase contrast, differential interference contrast, phase-imaging, magnetic resonance imaging, Raman scattering imaging, or a combination thereof.

In some embodiments, the method can comprise linking the sequence data (e.g. a profile) of the first cell and/or the second cell in the partition with the characteristics of the first cell and/or the second cell in the partition, the phenotypic feature of the first cell and/or the second cell, and/or the interaction of interest between the first cell and the second cell using the cell barcode sequence of the retained barcoded nucleic acid and the cell barcode sequence of the pooled barcoded nucleic acid in the sequencing data.

Therefore, by attaching identical cell barcodes to sample nucleic acids of one or more interacting cells within a given partition and matching such cell barcodes of with the cell barcodes associated with the partition, the sequences of the pair of interacting cells having the identical cell barcodes can be tracked to that partition. Cell characteristics such as phenotypic observables (e.g. cellular metabolism, cell cycle states, cell signaling, cell viability etc.) of the interacting cells can be obtained. Cell nucleic acid sequences or a profile to the cell functionality and the nature of the cell-cell interaction can thus be linked.

### An Embodiment of a Method for Determining Cell-Cell Interaction

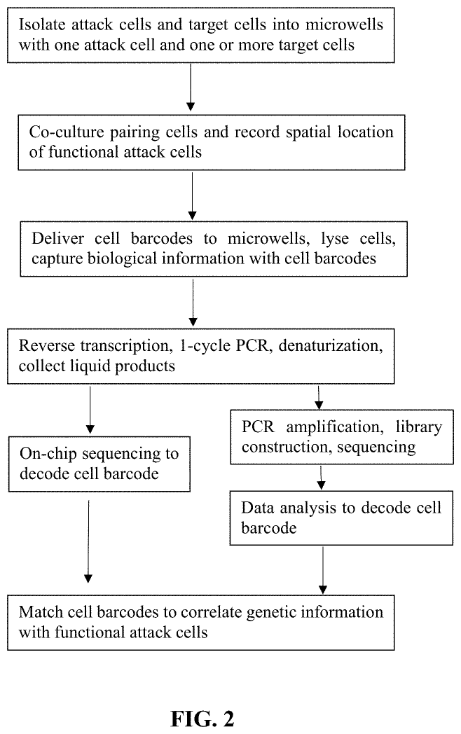

FIG. 2 shows a non-limiting embodiment of a method described herein for determining cell-cell interaction.

In the exemplary embodiment, a plurality of first cells (e.g. attack cells) and a plurality of second cells (e.g. target cells) are introduced into microwells of a microwell array. The microwells comprise a microwell containing one attack cell and one or more target cells. The attack cell can potentially attack or be silent to the target cell. The pairing cells (e.g. the attack cell and the one or more target cells) can be cocultured to allow them to interact. A spatial location of functional attack cells can be recorded. The functional attack cells can be attack cells exhibiting certain phenotypic features, such as inhibiting the viability of the one or more target cells. Barcode molecules comprising cell barcode sequences are delivered to the microwells. Cells are lysed in the microwells, allowing the barcode molecules to capture biological information, i.e. sample nucleic acids associated with the attack cell and the one or more target cells. The sample nucleic acids are barcoded. Barcoding can be performed as illustrated in FIGS. 1A-1B. RNAs are reversed transcribed to first-strand cDNA comprising a barcode molecule and a sample nucleic acid. The first-strand cDNA is then converted into double-stranded cDNA comprising the barcode molecule and the sample nucleic acid by one-cycle PCR. Double-stranded barcoded nucleic acids are denaturized to generate one single-stranded barcoded nucleic acid retained in the microwells and the other single-stranded barcoded nucleic acid which can be pooled by collecting the liquid products in the microwells. The pooled single-stranded barcoded nucleic acids can be amplified and undergo library construction processes followed by sequencing to obtain nucleic acid sequences of the pooled barcoded nucleic acids. The nucleic acid sequences of the pooled barcoded nucleic acids can be analyzed to decode cell barcode sequences. The single-stranded barcoded nucleic acid retained in the microwells can be sequenced on-chip to decode cell barcode sequences. The cell barcode sequences of the pooled barcoded nucleic acids and the cell barcode sequences of the retained barcoded nucleic acid sequences can be matched to correlate the genetic information with the functional attack cells.

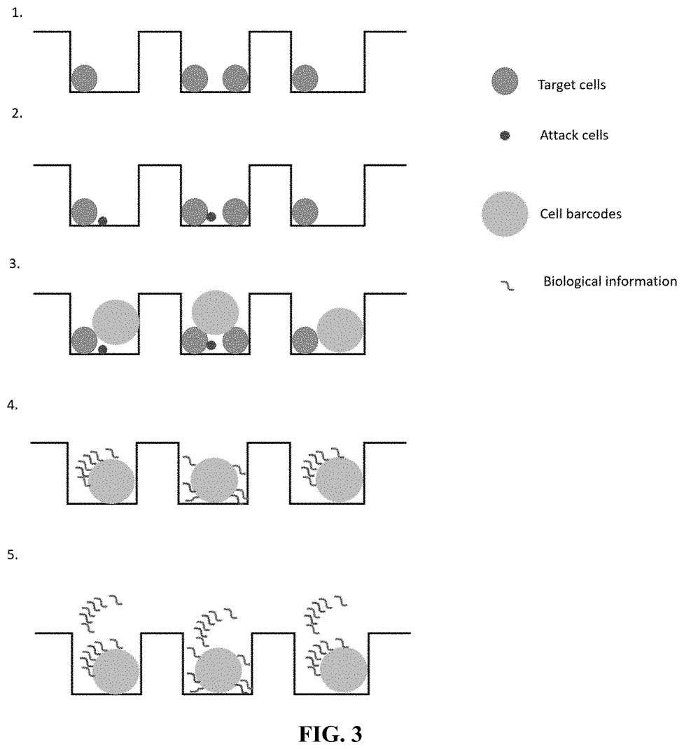

FIG. 3 shows a process schematic of an embodiment disclosed herein. Target cells are partitioned into microwells of a microwell array (1). Attack cells are also partitioned into the microwell array to have one attack cell with one or more target cells (2). Some microwells only have target cells but no attack cell. Cells can be co-cultured and spatial location information of attack cells having a desired functionality can be recorded. Beads carrying barcode molecules are then delivered to the microwells to have at most one bead per microwell (3). Cells are lysed to release the sample nucleic acids associated with the target and/or the attack cells. The sample nucleic acids are barcoded (4). The barcoded nucleic acids attached to the beads are retained in the microwells (5). The barcoded nucleic acids released to the solution are pooled into a pooled mixture outside the microwell array (5). Both barcoded nucleic acids are subjected to a sequencing process. For example, the barcoded nucleic acids retained in the microwells can be sequenced using the on-chip sequencing process described in Example 1. The barcoded nucleic acids pooled in bulk can be sequenced using any suitable sequencing method described herein. The barcode sequences of the barcoded nucleic acids retained in the microwell array and the barcode sequences of the pooled barcoded nucleic acids can be matched to correlate genetic information with attack cells with a desired functionality.

The method disclosed herein can be used in the analysis of gene expression of a cell, including the identification of RNA transcripts and their quantitation to detect changes in gene expression upon cell-cell interaction. The method can be used to analyze the RNA transcripts present in an individual cell alone and the RNA transcripts present in an individual cell in contact with another cell. For example, when the first cell and the second cell interact, the interaction can increase or decrease the expression level of one or more genes in the first cell or the second cell with respect to the expression level of the one or more genes in the first cell or the second cell alone.

### EXAMPLES

Some aspects of the embodiments discussed above are disclosed in further detail in the following examples, which are not in any way intended to limit the scope of the present disclosure.

### Example 1

### An Exemplary Method for Determining Cell-Cell Interaction

### Microfluidic Device Fabrication

The microfluidic devices were made from polydimethylsilioxane (PDMS). In brief, a silicon wafer with array patterning of micro-columns (45 um in diameter, 45 um in height, and 30 um in spacing) was used as a mold. PDMS (Sylgard 184) was cast over the silicon mold and cured at 80° C. for 1 hour. Then the cured PDMS was peeled from the silicon mold with microwell array on its surface. This PDMS with microwell array was used as a substrate, and bonded with another piece of PDMS with a flow channel to form a closed format microfluidic device. The flow channel has an inlet and an outlet. Samples and reagents can be perfused from the inlet through the flow channel to deliver to the microwell array, and the waste can be pushed out from the outlet and removed.

In this example, a microwell array having 30,000 to 50,000 microwells was used. The number of microwells depends on the size of the flow channel.

### Microfluidic Device Priming

The microfluidic device was primed before usage to increase the surface hydrophilicity. 100% ethanol was used to flush the surface and remove air bubbles trapped in the microwells. Then priming solution was perfused into the flow channel and treat the microwell array surface for at least 30 minutes at room temperature. A degassing process by vacuum (15 min) might be added after replacing the 100% ethanol with the priming solution. After priming, perfuse the microfluidic flow channel with Phosphate Buffered Saline (PBS) solution to remove the priming solution.

### Preparation of Cells

The NIH/3T3 mouse fibroblast cells and K562 human leukemia cells were cultured in DMEM with 10% Fetal Bovine Serum (FBS) and penicillin and streptomycin (PS) at 37° C. with 5% CO2. Cells were collected from cell culture flask and centrifuged at 500 g for 3 minutes to form cell pellet. The cell pellet was resuspended in 1 mL of PBS with RNase Inhibitor. The cell suspension was passed through a 40 um cell strainer to remove cell clusters. Then the cells were counted under hemocytometer to dilute to 1×10⁵ cells/mL. For mixed cell experiments, 3T3 cells and K562 cells were mixed at 1:1 ratio.

### Cell Loading

About 100 uL single cell suspension was loaded from the inlet into the microfluidic device. The concentration of single cell suspension was 1-3*10⁵ cells/mL to guarantee about 10% cell coverage of microwell array with low doublets (more than one cell in one microwell). Then the extra liquid was removed from both the inlet and the outlet of the microfluidic device to stop the flow inside the flow channel. The cells in the suspension gradually precipitated into the microwells due to the gravity. After several minutes, PBS solution was flushed through the flow channel to remove extra cells not precipitating inside the microwells.

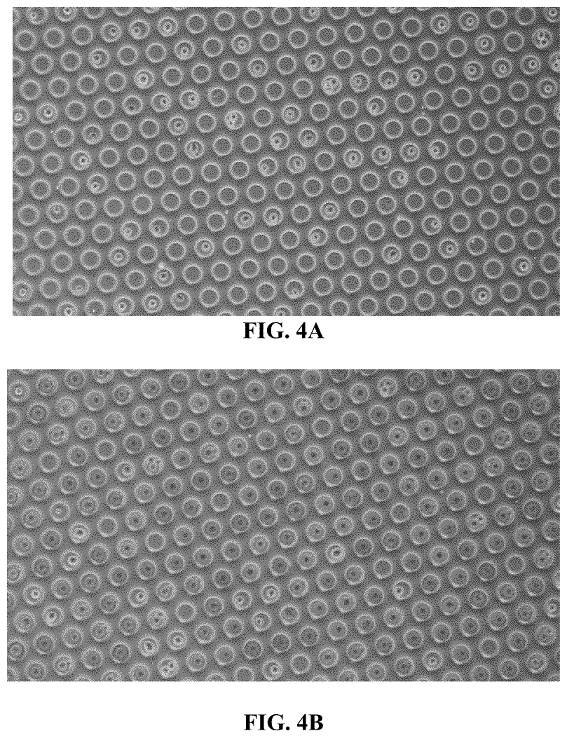

FIG. 4A demonstrates an exemplary microwell array loaded with about 10% single cell coverage of the microwells.

### Bead Loading

Barcoded microbeads (about 80,000 beads in 50 uL) were slowly loaded into the microfluidic device to cover halfway of the flow channel. Then, 100 uL PBS was perfused to slowly push the microbeads to move forward and to completely cover the microwells inside the whole flow channel. Ensure that >80% microwell arrays were covered with microbeads. Excess beads were washed out with PBS. The microbeads had a diameter about 30 um to ensure that only one bead can fall into one microwell.

FIG. 4B demonstrates an exemplary microwell array loaded with microbeads to ensure only one microbead in one microwell.

Cell Lysis and mRNA Capture

Cell lysis buffer was perfused into the device to lyse the cells. Then the device was incubated at room temperature for 30 minutes letting mRNA released from cells to bind to the barcodes on the microbeads.

### On-Chip Reverse Transcription and 1-Cycle Polymerase Chain Reaction

After mRNA binding, washing buffer was perfused through the flow channel. Then reverse transcription (RT) mixer solution with template switching primers with an identical sequence was loaded into the device. The device was incubated at room temperature for 30 min, and then at 42° C. for 90 min to finish the RT reaction. After RT reaction, NaOH solution was used to denaturize the formed half double strand cDNA. After washing, Polymerase Chain Reaction (PCR) solution was loaded into the device and the device was kept at 65° C. for about 3 min for annealing and 72° C. for about 5-10 min for extension. After the 1-cycle PCR reaction, NaOH solution was used to denaturize the formed completed double strand cDNA. In this case, the liquid solution inside the microfluidic flow channel was collected and neutralized with HCl for next step of purification and PCR amplification. The microfluidic device with barcode microbeads filled microwell array was filled with buffer and kept for on-chip sequencing.

### Purification and PCR Amplification

This section demonstrates the capture, purification and PCR amplification of the cDNA collected in the liquid solution from the microfluidic device.

The collected liquid solution from the microfluidic device has floating cDNA. cDNA with desirable sequence length (i.e., ˜1000 bp) was selectively captured by magnetic beads. Then the magnetic beads with selected cDNA was mixed with PCR solution and PCR was carried out to amplify the captured cDNA.

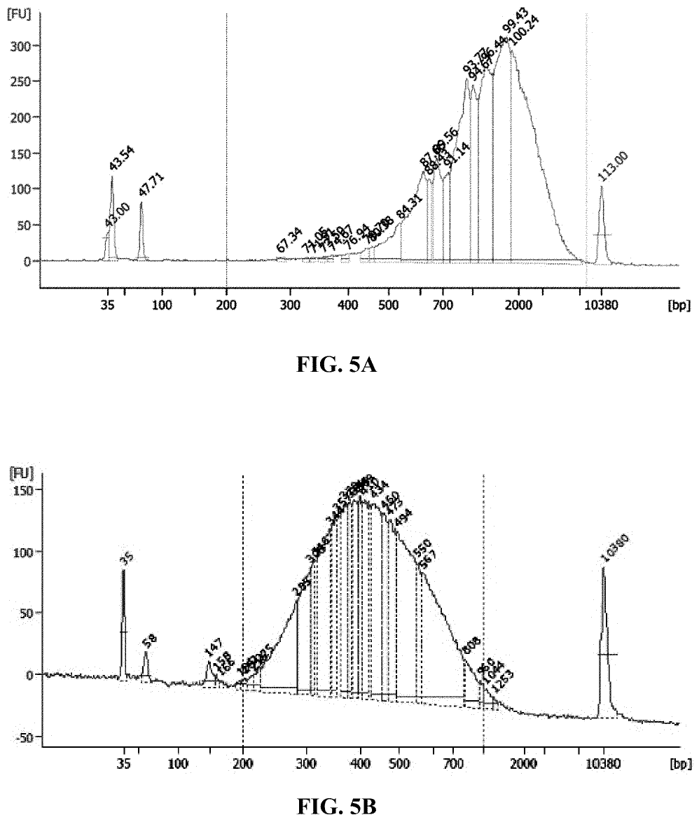

FIG. 5A is a non-limiting exemplary trace showing the quality of the purified cDNA solution after PCR amplification.

### Library Construction and Sequencing

The cDNA in the purified solution was then inputted for standard Nextera tagmentation and amplification reactions to construct libraries for sequencing. A custom primer instead of i5 index primer was used to amplify only those fragments containing the cell barcodes and UMIs. The library product was then purified again using magnetic beads. The cDNA concentration of the libraries was measured by Fluorometer and the quality of the libraries was characterized by Agilent Bioanalzyer 2100 (FIG. 5B). FIG. 5B is a non-limiting exemplary trace the quality of the purified libraries. The libraries were sequenced on HiSeq 2500 sequencer using a custom primer for Read 1.

### Sequencing Data Analysis

Following the sequencing step described above, transcriptome alignment including barcode/UMI identification was performed by using DropSeq tools. In brief, the Read 2 sequencing data included cell barcode and UMI sequences, while the Read 1 sequencing data contained the transcript information of captured mRNA. The reads were aligned to reference transcriptome of the corresponding species (mouse; human; human-mouse mix) to extract gene expression matrix.

Further data analysis was performed by using Seurat. The cell number was determined by selecting the cells with: (1) UMI number was larger than 1000; (2) gene number was between 500 and 8000; and (3) reads were larger than 10000. In this experiment, the estimated cell number was about 200, with median genes being about 1000 and median UMI being about 2000.

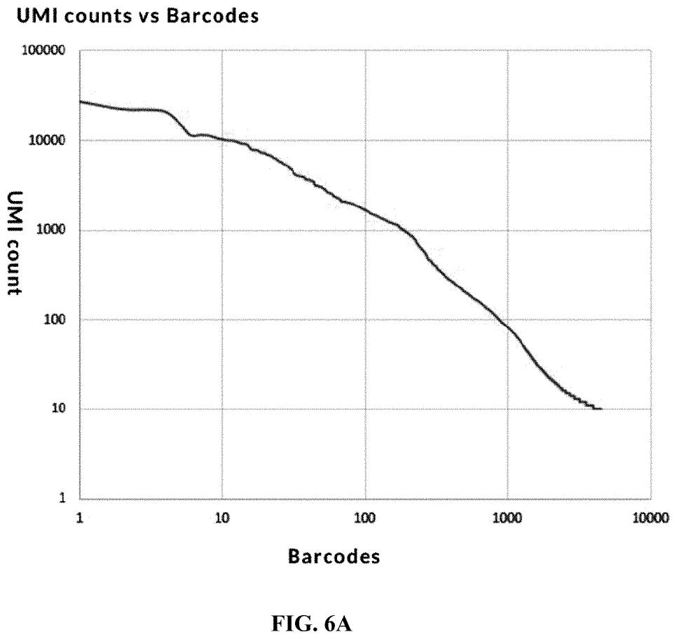

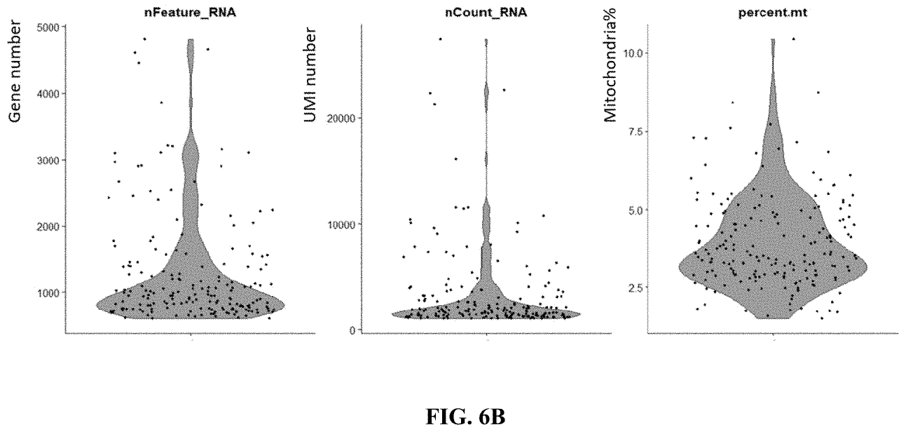

FIG. 6A shows one non-limiting exemplary plot of the UMI count vs. cell barcode number.

FIG. 6B depicts Violin plots of the gene number, UMI number, and mitochondria percentage distribution of determined cells.

### On-Chip Sequencing

This section demonstrates the on-chip sequencing process performed on the microfluidic device filled with the barcoded microbeads. The on-chip sequencing was performed using the automatic flow control system described in Example 2.

On-chip sequencing was performed by using Sequencing by Ligation (SBL) method to decode the cell barcode sequence of each microbead. In this design, the cell barcode was a 12 bp sequence and was same for the same microbeads, but was different for different microbeads. The total sequencing process for decoding the cell barcode sequence included 15 rounds, with 3 rounds of sequencing using each of five sequencing primers. The sequencing primers, i.e., primers N-0, N-1, N-2, N-3, and N-4, were 21 bp oligonucleotide sequences complementary to partial sequence of the PCR handle sequence on the microbeads (Table 1). The PCR handle sequence had a total 25 bp. The N-0 primer was complementary to the last 21 bp of the PCR handle sequence from the end. The N-1 primer was complementary to the second from last 21 bp of the PCR handle sequence. The N-2 primer was complementary to the third from last 21 bp of the PCR handle sequence. The N-3 primer was complementary to the fourth from last 21 bp of the PCR handle sequence. The N-4 primer was complementary to the fifth from last 21 bp of the PCR handle sequence, or in the other way, was complementary to the first 21 bp of the PCR handle sequence from the beginning.

TABLE 1
Oligonucleotide sequences of PCR handle
and Sequencing Primers N-0, N-1,
N-2, N-3, and N-4.

| | | SEQ |
|---|---|---|
| | | ID |
| | Sequence | NO |
| PCR handle | AAG CAG TGG TAT | 1 |
| | CAA CGC AGA GTA | |
| | C | |
| Primer N0 | TC ACC ATA GTT | 2 |
| | GCG TCT CAT G | |
| Primer N1 | GTC ACC ATA GTT | 3 |
| | GCG TCT CAT | |
| Primer N2 | C GTC ACC ATA | 4 |
| | GTT GCG TCT CA | |
| Primer N3 | TC GTC ACC ATA | 5 |
| | GTT GCG TCT C | |
| Primer N4 | TTC GTC ACC ATA | 6 |
| | GTT GCG TCT | |

During on-chip sequencing, first, the sequencing device was perfused with priming buffer to get the system ready for on-chip sequencing process. Next, a mixture of N-0 sequencing primer with sodium acetate, tri-sodium citrate, acetic acid solution was loaded into the sequencing device to replace the priming buffer. The sequencing device was incubated for 10 minutes at room temperature. During incubation, the N-0 sequencing primer hybridizes to the last 21 nucleotide sequence of the PCR handle sequence, just stops at before the cell barcode sequence. Then three rounds of sequencing were subsequently performed to add fluorescent dye modified sequencing oligos to decode the cell barcode sequence. Each sequencing oligo has a dibase sequence, 3 degenerated bases, 3 universal bases, and a fluorescent dye from 3′ end to the 5′ end. The dibase sequence was the 16 combination of dibases of A, T, C, and G. In total 16 different fluorescent dye modified sequencing oligos were used Table 2).

TABLE 2
Sequences of sequencing
oligonucleotides.

| SBL fluorescent | | | |
|---|---|---|---|
| dye: | | | SEQ |
| 3′-dibases- | | | ID |
| NNNUUU(dye)-5′ | Dye | Dibases | NO |
| AANNNUUU-FAM | FAM | AA | 7 |
| CCNNNUUU-FAM | FAM | CC | 8 |
| GGNNNUUU-FAM | FAM | GG | 9 |
| TTNNNUUU-FAM | FAM | TT | 10 |
| CANNNUUU-Cy3 | Cy3 | CA | 11 |
| ACNNNUUU-Cy3 | Cy3 | AC | 12 |
| TGNNNUUU-Cy3 | Cy3 | TG | 13 |
| GTNNNUUU-Cy3 | Cy3 | GT | 14 |
| GANNNUUU-TXR | TXR | GA | 15 |
| AGNNNUUU-TXR | TXR | AG | 16 |
| TCNNNUUU-TXR | TXR | TC | 17 |
| CTNNNUUU-TXR | TXR | CT | 18 |
| TANNNUUU-Cy5 | Cy5 | TA | 19 |
| ATNNNUUU-Cy5 | Cy5 | AT | 20 |
| CGNNNUUU-Cy5 | Cy5 | CG | 21 |
| GCNNNUUU-Cy5 | Cy5 | GC | 22 |

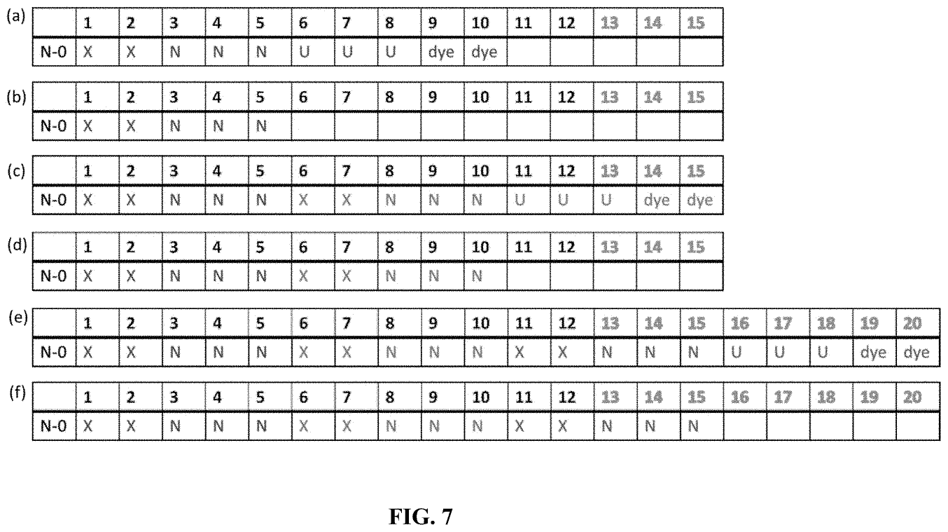

Each round of sequencing includes the following steps: (1) A ligation reaction, in which the sequencing device was incubated with ligation mixture and sequencing oligos for 45 minutes to let the first sequencing oligos ligate to the cell barcode; (2) An imaging process, in which the sequencing device was imaged under four different fluorescent color: GFP, RFP, Texas Read, and Cy5, after the device was washed with the instrument buffer for 5 minutes, and filled with imaging buffer; (3) A fluorophore cleavage process, in which the sequencing device was incubated with the Cleave Solution 1 for 5 minutes and the reconstituted Cleave Solution 2 for another 5 minutes to remove the 3 universal bases and the fluorescent dye of the ligated first sequencing oligos. In the first round of sequencing, the first two bases of the cell barcode sequence were decoded. Then the second round of sequencing repeats these three steps to ligate a second sequencing oligo to the cell barcode sequence, to image, and to cleave the part of universal bases and fluorescent dyes and make space available for next sequencing oligos. In the second round of sequencing, the sixth and the seventh bases of the cell barcode sequence were decoded. And the third round of sequencing was performed accordingly to decode the eleventh and twelfth bases of the cell barcode sequence (FIG. 7).

FIG. 7 illustrates the three rounds of sequencing for primer N-0. Step (a) shows the ligation of the first sequencing oligos to the 12 bp cell barcode sequences in the first round of sequencing; Step (b) shows the cleavage of the 3 universal bases and fluorescent dye of the first sequencing oligos, only the dibase sequence and the 3 degenerated bases of the first sequencing oligo left ligated to the cell barcode sequences; step (c) shows ligation of the second sequencing oligos to the 12 bp cell barcode sequences in the second round of sequencing; step (d) shows cleavage of the 3 universal bases and fluorescent dye of the second sequencing oligos, only the dibase sequence and the 3 degenerated bases of the second sequencing oligos left ligated to the cell barcode sequences; step (e) shows ligation of the third sequencing oligos to the 12 bp cell barcode sequences in the third round of sequencing; step (f) shows cleavage of the 3 universal bases and fluorescent dye of the third sequencing oligos, only the dibase sequence and the 3 degenerated bases of the third sequencing oligos left ligated to the cell barcode sequences.

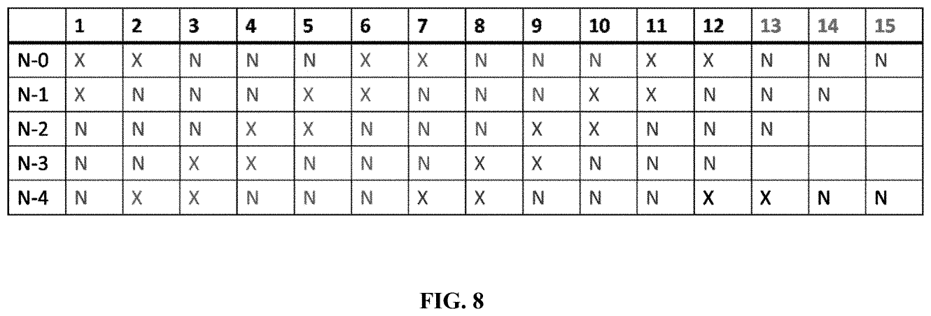

After three rounds of sequencing, the sequencing primers N-0 were stripped by continuously flowing strip solution through the sequencing device for 10 minutes. Then a mixture of N-1 sequencing primers with sodium acetate, tri-sodium citrate, acetic acid solution was loaded into the sequencing device to hybridize the sequencing primers N-1 to the barcodes. Another three rounds of sequencing were repeated to decode the first, fifth, sixth, tenth, and eleventh bases of the cell barcode sequences. Similarly, the hybridizations of sequencing primers N-2, N-3, and N-4 were performed subsequently with three sequencing rounds for each sequencing primers. However, for the sequencing primer N-4, there were four rounds to ligate the sequencing oligos, but only the last three rounds were sequenced (FIG. 8).

FIG. 8 illustrates the decoding process of the 12 bp cell barcode sequence for five sequencing primers. After the total 15 sequencing rounds, all 12 bp of the cell barcode sequences were decoded with each base being sequenced twice to reduce the sequencing error.

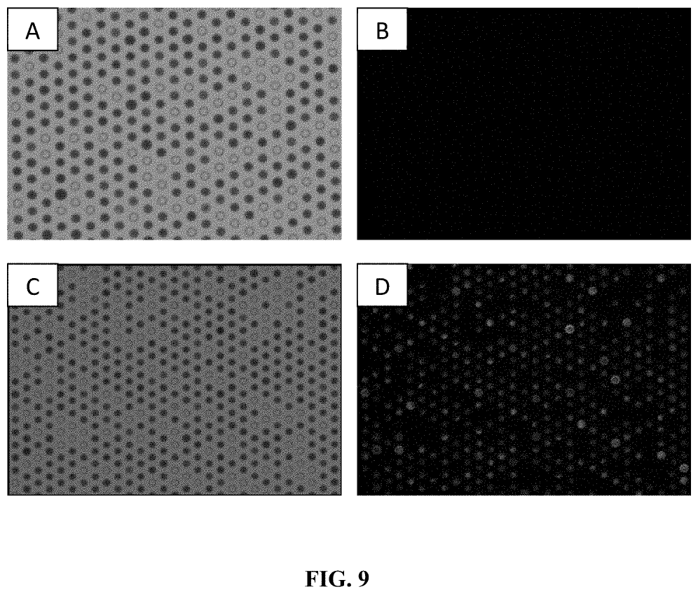

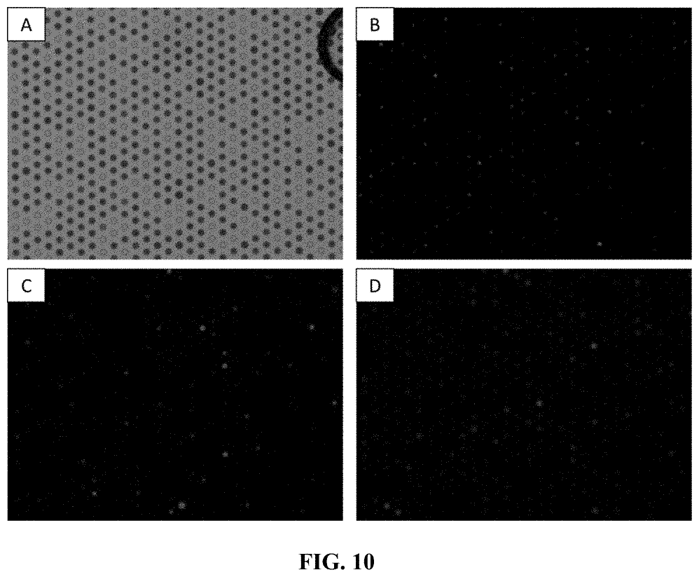

FIG. 9 shows, in a non-limiting example, the microbeads with barcodes inside a microfluidic device with a microwell array before adding the sequencing primer N-0 (in panels A and B) and after adding the sequencing primer N-0 (in panels C and D). Panel A: bright field imaging of microbeads; Panel B: fluorescent imaging of microbeads under filter Cy5; Panel C: bright field imaging of microbeads; and Panel D: fluorescent imaging of microbeads under filter Cy5. The sequencing primer N-0 was modified with a Cy5 fluorescent dye at its end, thus the sequencing primer N-0 was visible under fluorescent microscope. Before adding the sequencing primer N-0, there was no fluorescent signal coming from the microbeads as shown in panels A and B. After adding the sequencing primer N-0, almost all beads show fluorescent signals as shown in panels C and D.

FIG. 10 shows, in a non-limiting example, the microbeads with barcodes inside a microfluidic device with a microwell array after adding a mixture of four sequencing oligos with fluorescent dye of GFP/FAM, RFP/Cy3, Texas Red, and Cy5 following the addition of sequencing primer N-0 during an on-chip sequencing process. Panel A: bright field imaging of microbeads; Panel B: fluorescent imaging of microbeads under fluorescent filter GFP/FAM (green); Panel C: fluorescent imaging of microbeads under fluorescent filter RFP/Cy3 (red); and Panel D: fluorescent imaging of microbeads under fluorescent filter Cy5 (magenta). The four fluorescent oligos correspond to the dibase sequences: CA (RFP/Cy3), CT (Texas Red), CC (GFP/FAM), CG(Cy5). The microbeads with complementary sequences to the dibase sequences adjacent to the PCR primer were visible under different fluorescent filters, indicating the binding of sequencing oligos to the microbead barcodes. There were a total of 16 different combinations of dibase sequences, while only 4 dibase sequences are demonstrated in FIG. 10. Thus, there were about one quarter of all microbeads visible under fluorescent microscope.

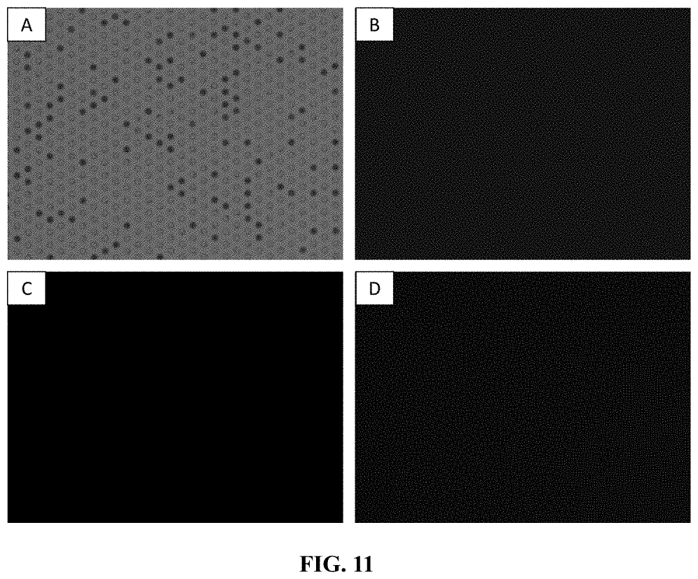

FIG. 11 shows, in a non-limiting example, the microbeads with barcodes inside a microfluidic device with a microwell array after cleavage of the sequencing oligos with fluorescent dye of GFP, RFP, Texas Read and Cy5. Panel A: bright field imaging of the microbeads; Panel B: fluorescent imaging of the microbeads under fluorescent filter GFP (Green); Panel C: fluorescent imaging under fluorescent filter RFP (Red); and Panel D: fluorescent imaging under fluorescent filter Cy5 (magenta). There was no signal under the fluorescent filter GFP (panel B), RFP (panel C), and Cy5 (panel D). This indicates the complete removal of the fluorescent dyes on the fluorescent sequencing oligos, rendering the barcodes ready for the addition of next round of fluorescent sequencing oligos.

### Example 2

### Automatic Flow Control System for On-Chip Sequencing

This example describes an automatic flow control system used herein for the on-chip sequencing of Example 1. In this example, the microfluidic device with barcoded microbeads filled microwell array is connected to an automatic flow control system for the subsequent on-chip sequencing.

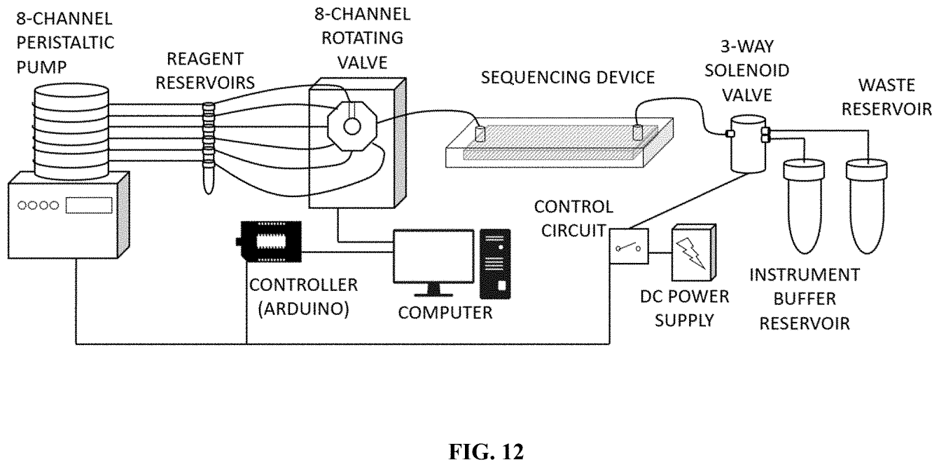

An automatic system is set up to use computer to automate the on-chip sequencing process. The automatic system includes a computer, a peristaltic pump, a rotating valve system, a controller, a sequencing device, solenoid valves, reagent reservoirs, waste reservoir, instrument buffer reservoir, and a power supply (FIG. 12). The sequencing device is a microfluidic device filled with barcoded microbeads. The sequencing device is connected with an 8-channel rotating valve through its inlet and with a 3-way solenoid valve through its outlet.

FIG. 12 shows a schematic of an automatic flow control system used for on-chip sequencing. Each channel of the rotating valve is connected to one reagent reservoir holding a specific reagent. The rotating valve is used to changing the reagent connected to the inlet of the sequencing device by switch its rotating head. The reagent reservoirs are pressurized by an 8-channel peristaltic pump. One reagent reservoir has one inlet airtight sealed with a connecting tube from the peristaltic pump and one outlet airtight sealed with a connecting tube from the rotating valve. The opening of the connecting tube from the peristatic pump is set above the surface of reagent, while the opening of the connecting tube from the rotating valve is set at the bottom of the reservoir.

The outlet of the sequencing device is connected to a waste reservoir and an instrument buffer reservoir through a 3-way solenoid valve. If the peristaltic pump provides a positive pressure to a reagent reservoir, the reagent in the reservoir will be pushed up through the 8-channel rotating valve and perfused into the inlet of the sequencing device. In this case, the solution replaced by the reagent from the sequencing device is send to the waste reservoir by opening the solenoid valve connect to the waste reservoir. While if the peristaltic pump provides a negative pressure, the reagent in the sequencing device will be withdrawn from the inlet through the 8-channel rotating valve and back to the reagent reservoir. Under this condition, the solenoid valve switches on the connection to the instrument buffer reservoir and lets the instrument buffer flush into the sequencing device through the outlet. The positive and negative of the pressure provided by the peristaltic pump is controlled by the rotation direction of the peristaltic pump. Additionally, the flow rate of each reagent can be altered by changing the diameter of connecting tubes. The rotation direction and the on/off of the peristaltic pump, and the controller of the solenoid valve, are controlled by a computer through an ARDUINO controller. The switch of channel of rotating valve is directly controlled by the computer.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of determining an expression profile of a cell involved in a cell-to-cell interaction of interest, comprising:
partitioning a plurality of first cells and a plurality of second cells into a plurality of partitions;
determining an interaction of interest between one first cell of the plurality of first cells and one second cell of the plurality of second cells in a partition of the plurality of partitions;
introducing a bead comprising a plurality of barcode molecules into the partition, wherein barcode molecules of the plurality of barcode molecules comprise an identical cell barcode and different unique molecule identifiers (UMIs);
barcoding a plurality of sample nucleic acids associated with the first cell and/or the second cell in the partition using the plurality of barcode molecules to generate a plurality of barcoded nucleic acids;
pooling a first subset of barcoded nucleic acids of the plurality of barcoded nucleic acids, whereby a second subset of barcoded nucleic acids of the plurality of barcoded nucleic acids are retained in the partition;
determining sequences of the pooled barcoded nucleic acids;
determining an expression profile from the sequences of the pooled barcoded nucleic acids;
determining a sequence of the identical cell barcode of the retained barcoded nucleic acids in the partition;
matching the sequence of the identical cell barcode of the retained barcoded nucleic acids and a sequence of the identical cell barcode in the sequences of the pooled barcoded nucleic acid;
determining an expression profile of the first cell and/or the second cell as the expression profile determined from the sequences of the pooled barcoded nucleic acids; and
matching the expression profile of the first cell and/or the second cell determined from the sequences of the pooled barcoded nucleic acids with the interaction of interest.

2. A method of nucleic acid sequencing, comprising:
(a) partitioning a plurality of first cells and a plurality of second cells into a plurality of partitions, wherein a partition of the plurality of partitions comprises one first cell of the plurality of first cells and one second cell of the plurality of second cells;
(b) introducing a plurality of barcode molecules to the partition, wherein each of the plurality of barcode molecules comprises a first barcode sequence and a second barcode sequence, and wherein the first barcode sequences of two barcode molecules of the plurality of barcode molecules are identical;
(c) barcoding a plurality of sample nucleic acids associated with the first cell and/or the second cell in the partition using the plurality of barcode molecules to generate a plurality of barcoded nucleic acids;
(d) sequencing the plurality of barcoded nucleic acids to obtain nucleic acid sequences of the plurality of barcoded nucleic acids; and
(e) determining a location of the partition the plurality of sample nucleic acids associated with the first cell and/or the second cell being barcoded is present using the first barcode sequences determined in the nucleic acid sequences of two barcoded nucleic acids of the plurality of barcoded nucleic acids.

3.-8. (canceled)

9. The method of claim 2, wherein introducing the plurality of barcode molecules to the partition comprises: introducing a plurality of extension primers to the partition, and wherein barcoding the plurality of sample nucleic acids comprises:
(c1) extending the plurality of extension primers using the plurality of sample nucleic acids as templates and the plurality of barcode molecules as template switching oligonucleotides to generate a plurality of single-stranded barcoded nucleic acids of the plurality of barcoded nucleic acids that are hybridized to the plurality of sample nucleic acids; and
(c2) generating a plurality of double-stranded barcoded nucleic acids of the plurality of barcoded nucleic acids using the plurality of single-stranded barcoded nucleic acids as templates.

10.-15. (canceled)

16. The method of claim 2, wherein the plurality of sample nucleic acids comprises ribonucleic acid (RNA).

17.-20. (canceled)

21. The method of claim 2, comprising pooling barcoded nucleic acids of the plurality of barcoded nucleic acids, after barcoding the plurality of sample nucleic acids and before sequencing the plurality of barcoded nucleic acids, to obtain pooled barcoded nucleic acids.

22.-29. (canceled)

30. The method of claim 21, comprising performing a polymerase chain reaction in bulk, subsequent to pooling barcoded nucleic acids of the plurality of barcoded nucleic acids, on the pooled barcoded nucleic acids, thereby generating amplified barcoded nucleic acids.

31.-35. (canceled)

36. The method of claim 2, wherein the plurality of barcoded nucleic acids comprises barcoded nucleic acids retained in the partition, and wherein sequencing the plurality of barcoded nucleic acids comprises sequencing the barcoded nucleic acids retained in the partition.

37. (canceled)

38. (canceled)

39. The method of claim 36, wherein sequencing the barcoded nucleic acids retained in the partition comprises: determining the first barcode sequences of the barcoded nucleic acids retained in the partition using oligonucleotide probes each comprising a fluorescent label.

40.-44. (canceled)

45. The method of claim 2, wherein the two barcoded nucleic acids of the plurality of barcoded nucleic acids comprise a retained barcoded nucleic acid of the retained barcoded nucleic acids and a pooled barcoded nucleic acid of the pooled barcoded nucleic acids, and wherein the first barcode sequence of the retained barcoded nucleic acid and the first barcode sequence of the pooled barcoded nucleic acid are identical.

46. The method of claim 2, wherein determining the location of the partition the plurality of sample nucleic acids associated with the first cell and/or the second cell being barcoded is present comprises: matching the first barcode sequence of the barcoded nucleic acids retained in the partition determined and the first barcode sequence of the pooled barcoded nucleic acids in the nucleic acid sequences obtained.

47. The method of claim 46, comprising: recording a location of the partition comprising the first cell and the second cell, or characteristics of the first cell and/or the second cell therein, to provide a recorded location of the partition, or characteristics of the first cell and/or the second cell therein.

48. The method of claim 47, comprising: linking the recorded location of the partition comprising the first cell and the second cell, or the characteristics of the first cell and/or the second cell therein, with the first barcode sequence of the barcoded nucleic acids retained in the partition determined, thereby linking the recorded location of the partition with the first barcode sequence of the pooled barcoded nucleic acids associated with the first cell and/or the second cell.

49. The method of claim 47, wherein recording the location of the partition comprising the first cell and the second cell, or characteristics of the first cell and the second cell therein, comprises optically imaging the partition, or the first cell and the second cell therein.

50. The method of claim 47, wherein the characteristics of the first cell and/or the second cell therein comprises a phenotypic feature of the first cell and/or the second cell.

51. (canceled)

52. (canceled)

53. The method of claim 2, wherein an interaction between the first cell and the second cell is of interest.

54.-58. (canceled)

59. The method of claim 2, comprising:
determining a profile of the first cell and/or the second cell using the nucleic acid sequences of the plurality of barcoded nucleic acids, optionally wherein determining the profile of the first cell and/or the second cell comprises: determining the profile of the first cell and/or the second cell using the second sequences and sequences of the sample nucleic acids, or a portion thereof, present in the nucleic acid sequences.

60. The method of claim 59, wherein the expression profile of the first cell and/or the second cell in the partition determined is different from an expression profile of the first cell or the second cell alone.

61. The method of claim 59, comprising
linking the profile of the first cell and/or the second cell in the partition determined with the characteristics of the first cell and/or the second cell in the partition, the phenotypic feature of the first cell and/or the second cell, and/or the interaction of interest between the first cell and the second cell using the first barcode sequence of the retained barcoded nucleic acid determined and the first barcode sequence of the pooled barcoded nucleic acid in the sequencing data.

62. The method of claim 2, comprising:
culturing the first cell and the second cell in the partition subsequent to partitioning the plurality of first cells and the plurality of second cells and prior to barcoding the plurality of sample nucleic acids.

63.-84. (canceled)

85. The method of claim 2, wherein the plurality of partitions comprises a plurality of microwells, wherein a microfluidic device comprises the plurality of microwells, wherein the microfluidic device comprises an inlet port and/or an outlet port in fluid communication with the plurality of microwells, wherein partitioning the plurality of first cells and a plurality of second cells comprises flowing a solution comprising the plurality of first cells and/or the plurality of second cells into the plurality of microwells via the inlet port, and/or wherein flowing the solution comprising the plurality of first cells and/or the plurality of second cells comprises flowing a solution comprising the plurality of first cells and a solution comprising the plurality of second cells concurrently or sequentially.

86.-95. (canceled)
